SEQUENCE LISTING (196 sequences)

SEQ ID NO: 1 - DNA, 369 bp, synthetic construct
  source 1..369: mol_type = other DNA
caggtgcagctggtggagtctgggggaggcgtggtccagcctgggaggtccctgagactctcctgtgcagcctctggattcaccttcagtagttatggcatgcactgggtccgccaggctccaggcaaggggctggagtgggtggcagttatatcatatgatggaagtaataaatactatgcagactccgtgaagggccgattcaccatttccagagacaattccaagaacacgctgtatctgcaaatgaacagcctgagagctgaggacacggctgtgtattactgtgcgaaagtgggagctactactttctactactactacggtatggacgtctggggccaagggaccacggtcaccgtctcctca

SEQ ID NO: 2 - protein, 123 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKVGATTFYYYYGMDVWGQGTTVTVSS

SEQ ID NO: 3 - DNA, 24 bp, synthetic construct
  source 1..24: mol_type = other DNA
ggattcaccttcagtagttatggc

SEQ ID NO: 4 - protein, 8 aa, synthetic construct
GFTFSSYG

SEQ ID NO: 5 - DNA, 24 bp, synthetic construct
  source 1..24: mol_type = other DNA
atatcatatgatggaagtaataaa

SEQ ID NO: 6 - protein, 8 aa, synthetic construct
ISYDGSNK

SEQ ID NO: 7 - DNA, 48 bp, synthetic construct
  source 1..48: mol_type = other DNA
gcgaaagtgggagctactactttctactactactacggtatggacgtc

SEQ ID NO: 8 - protein, 16 aa, synthetic construct
AKVGATTFYYYYGMDV

SEQ ID NO: 9 - DNA, 321 bp, synthetic construct
  source 1..321: mol_type = other DNA
gacatccagatgacccagtctccatcttccgtgtctgcatttgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaaaaggctaacagtttcccattcactttcggccctgggaccaaagtggatatcaaa

SEQ ID NO: 10 - protein, 107 aa, synthetic construct
DIQMTQSPSSVSAFVGDRVTITCRASQGISSWLAWYQQKPGKAPKLLIYAASSLQSGVPSRFSGSGSGTDFTLTISSLQPEDFATYYCQKANSFPFTFGPGTKVEIK

SEQ ID NO: 11 - DNA, 18 bp, synthetic construct
  source 1..18: mol_type = other DNA
cagggtattagcagctgg

SEQ ID NO: 12 - protein, 6 aa, synthetic construct
QGISSW

SEQ ID NO: 13-14 - skipped (2 entries)

SEQ ID NO: 15 - DNA, 27 bp, synthetic construct
  source 1..27: mol_type = other DNA
caaaaggctaacagtttcccattcact

SEQ ID NO: 16 - protein, 9 aa, synthetic construct
QKANSFPFT

SEQ ID NO: 17 - DNA, 369 bp, synthetic construct
  source 1..369: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcaaatgatggaagtaataaatactatgtagattccgtgaagggccgattcaccatgggcagagacaattccaagaacacgctgtatctccaaatgaacagcctgagagttgaggacacggctgtgtattactgtgcgaaaggggctggaactctttactactactactacggtatggacgtctggggccaagggaccacggtcaccgtctcctca

SEQ ID NO: 18 - protein, 123 aa, synthetic construct
QVQLVESGGGVVQPGRSLRLSCAASGFTFSSYGMHWVRQAPGKGLEWVAVISNDGSNKYYVDSVKGRFTMGRDNSKNTLYLQMNSLRVEDTAVYYCAKGAGTLYYYYYGMDVWGQGTTVTVSS

SEQ ID NO: 19 - DNA, 24 bp, synthetic construct
  source 1..24: mol_type = other DNA
ggattcaccttcagtagctatggc

SEQ ID NO: 20 - protein, 8 aa, synthetic construct
GFTFSSYG

SEQ ID NO: 21 - DNA, 24 bp, synthetic construct
  source 1..24: mol_type = other DNA
atatcaaatgatggaagtaataaa

SEQ ID NO: 22 - protein, 8 aa, synthetic construct
ISNDGSNK

SEQ ID NO: 23 - DNA, 48 bp, synthetic construct
  source 1..48: mol_type = other DNA
gcgaaaggggctggaactctttactactactactacggtatggacgtc

SEQ ID NO: 24 - protein, 16 aa, synthetic construct
AKGAGTLYYYYYGMDV

SEQ ID NO: 25 - DNA, 321 bp, synthetic construct
  source 1..321: mol_type = other DNA
gacatccagatgacccagtctccttccaccctgtctgcatctgtaggaaacagagtcaccatcacttgccgggccagtcaaagtattagtagctggttggcctggtatcaacaaaaaccagggaaagcccctaagttcctgatctataaggcgtctagtttagaaagtggggtcccatcaaggttcagcggcagtggatctgggacagaattcactctcaccatcaccagcctgcagcctgatgattttgcaacttattactgccaacagtacaatatttattcgtggacgttcggccaagggaccaaggtggaaatcaaa

SEQ ID NO: 26 - protein, 107 aa, synthetic construct
DIQMTQSPSTLSASVGNRVTITCRASQSISSWLAWYQQKPGKAPKFLIYKASSLESGVPSRFSGSGSGTEFTLTITSLQPDDFATYYCQQYNIYSWTFGQGTKVEIK

SEQ ID NO: 27 - DNA, 18 bp, synthetic construct
  source 1..18: mol_type = other DNA
caaagtattagtagctgg

SEQ ID NO: 28 - protein, 6 aa, synthetic construct
QSISSW

SEQ ID NO: 29-30 - skipped (2 entries)

SEQ ID NO: 31 - DNA, 27 bp, synthetic construct
  source 1..27: mol_type = other DNA
caacagtacaatatttattcgtggacg

SEQ ID NO: 32 - protein, 9 aa, synthetic construct
QQYNIYSWT

SEQ ID NO: 33 - DNA, 372 bp, synthetic construct
  source 1..372: mol_type = other DNA
caggtgcagctggtggagtctgggggaggcgtggtccagcctgggaggtccctgagactctcctgtgcagcctctggattcaccttcagtacctatggcatgcactgggtccgccaggctccaggcaaggggctggagtggatggcagttatatcatttgatagaggtaataaatactatgcagactccgtgaagggccgattcaccatctccagagacaattccaagaacacgctgtatctgcaaatgaacagcctgagagctgaggacacggctgtgtattactgtgcgaaaggggggggttcggggactttctactactactacggtatggacgtctggggccaagggaccacggtcaccgtctcctca

SEQ ID NO: 34 - protein, 124 aa, synthetic construct
QVQLVQSGGGVVQPGRSLRLSCAASGFTFSTYGMHWVRQAPGKGLEWMAVISFDRGNKYYADSVKGRFTISRDNSKNTLYLQMNSLRAEDTAVYYCAKGGGSGTFYYYYGMDVWGQGTTVTVSS

SEQ ID NO: 35 - DNA, 24 bp, synthetic construct
  source 1..24: mol_type = other DNA
ggattcaccttcagtacctatggc

SEQ ID NO: 36 - protein, 8 aa, synthetic construct
GFTFSTYG

SEQ ID NO: 37 - DNA, 24 bp, synthetic construct
  source 1..24: mol_type = other DNA
atatcatttgatagaggtaataaa

SEQ ID NO: 38 - protein, 8 aa, synthetic construct
ISFDRGNK

SEQ ID NO: 39 - DNA, 51 bp, synthetic construct
  source 1..51: mol_type = other DNA
gcgaaaggggggggttcggggactttctactactactacggtatggacgtc

SEQ ID NO: 40 - protein, 17 aa, synthetic construct
AKGGGSGTFYYYYGMDV

SEQ ID NO: 41 - DNA, 324 bp, synthetic construct
  source 1..324: mol_type = other DNA
gacatccagatgacccagtctccatcttccgtgtctgcatctgtaggaaacagagtcaccatcacttgtcgggcgagtcagggtattagcagctggttagcctggtatcaccagaaaccagggaaagtccctaaggtcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccattcactttcggccctgggaccaaagtggatatcaaacga

SEQ ID NO: 42 - protein, 108 aa, synthetic construct
DIQMTQSPSSVSASVGNRVTITCRASQGISSWLAWYHQKPGKVPKVLIYAASSLQSGVPSRFSGSGSGTDFTLTISSLQPEDFATYYCQQANSFPFTFGPGTKVEIKR

SEQ ID NO: 43 - DNA, 18 bp, synthetic construct
  source 1..18: mol_type = other DNA
cagggtattagcagctgg

SEQ ID NO: 44 - protein, 6 aa, synthetic construct
QGISSW

SEQ ID NO: 45-46 - skipped (2 entries)

SEQ ID NO: 47 - DNA, 27 bp, synthetic construct
  source 1..27: mol_type = other DNA
caacaggctaacagtttcccattcact

SEQ ID NO: 48 - protein, 9 aa, synthetic construct
QQANSFPFT

SEQ ID NO: 49 - DNA, 375 bp, synthetic construct
  source 1..375: mol_type = other DNA
caggtacagctgcagcagtcaggtccaggactggtgaaaccctcgcagaccctctcactcacctgtgccatctccggggacagtgtctctagcaacagtcctgcttggaactggatcaggcagtccccatcgagaggccttgagtggctgggaaggacatactacaggtccaagtggtataatgattatgcagtgtctgtgagaggtcgaataaccatcaacccagacacatccaataaccagttctccctacatctgaactctgtgactcccgaggacacggcgatgtattactgtgcaagagacaagggtctaacagctcgtccgacctactttgactactggggccagggaaccctggtcaccgtctcctca

SEQ ID NO: 50 - protein, 125 aa, synthetic construct
QVQLQQSGPGLVKPSQTLSLTCAISGDSVSSNSPAWNWIRQSPSRGLEWLGRTYYRSKWYNDYAVSVRGRITINPDTSNNQFSLHLNSVTPEDTAMYYCARDKGLTARPTYFDYWGQGTLVTVSS

SEQ ID NO: 51 - DNA, 30 bp, synthetic construct
  source 1..30: mol_type = other DNA
ggggacagtgtctctagcaacagtcctgct

SEQ ID NO: 52 - protein, 10 aa, synthetic construct
GDSVSSNSPA

SEQ ID NO: 53 - DNA, 27 bp, synthetic construct
  source 1..27: mol_type = other DNA
acatactacaggtccaagtggtataat

SEQ ID NO: 54 - protein, 9 aa, synthetic construct
TYYRSKWYN

SEQ ID NO: 55 - DNA, 45 bp, synthetic construct
  source 1..45: mol_type = other DNA
gcaagagacaagggtctaacagctcgtccgacctactttgactac

SEQ ID NO: 56 - protein, 15 aa, synthetic construct
ARDKGLTARPTYFDY

SEQ ID NO: 57 - DNA, 326 bp, synthetic construct
  source 1..326: mol_type = other DNA
gacatccagatgacccagtctccttccaccctgtctgcatctgtaggagacagagtcaccatcacttgccgggccagtcagagtattaattactggttggcctggtatcagcagaaaccagggaaagcccctaagccccttatctataaggcgtctagtttagaaagtggggtcccatcaaggttcagcggcagtggatctgggacagagttcactctcaccatcagcagcctgcagcctgacgattttacaacttattactgccaacagtataatagttattctccgacgttcggccaagggaccaaggtggaaatcaaacgaac

SEQ ID NO: 58 - protein, 108 aa, synthetic construct
DIQMTQSPSTLSASVGDRVTITCRASQSINYWLAWYQQKPGKAPKPLIYKASSLESGVPSRFSGSGSGTEFTLTISSLQPDDFTTYYCQQYNSYSPTFGQGTKLEIKR

SEQ ID NO: 59 - DNA, 18 bp, synthetic construct
  source 1..18: mol_type = other DNA
cagagtattaattactgg

SEQ ID NO: 60 - protein, 6 aa, synthetic construct
QSINYW

SEQ ID NO: 61-62 - skipped (2 entries)

SEQ ID NO: 63 - DNA, 27 bp, synthetic construct
  source 1..27: mol_type = other DNA
caacagtataatagttattctccgacg

SEQ ID NO: 64 - protein, 9 aa, synthetic construct
QQYNSYSPT

SEQ ID NO: 65 - DNA, 378 bp, synthetic construct
  source 1..378: mol_type = other DNA
gaggtgcagctggtggagtctgggggaggcgtgatacagcctggggggtccctgagactctcctgtgcagcctctggattcaccttcgatgattatgccatgaactgggtccgtcaaggtccagggaagggtctggagtgggtctctgccataagtggtgatggcggtagcacatactatgcagactcggtgaagggccgattcaccatctccagagacaacagcaaaaactccctgtatctgcaaatgaacagtctgagagctgaggacaccgcctttttttactgtgcaaaagatctccgtaatacgatttttggagtggttattcccgatgcttttgatatctggggccaagggacaatggtcaccgtctcttca

SEQ ID NO: 66 - protein, 126 aa, synthetic construct
EVQLVESGGGVIQPGGSLRLSCAASGFTFDDYAMNWVRQGPGKGLEWVSAISGDGGSTYYADSVKGRFTISRDNSKNSLYLQMNSLRAEDTAFFYCAKDLRNTIFGVVIPDAFDIWGQGTMVTVSS

SEQ ID NO: 67 - DNA, 24 bp, synthetic construct
  source 1..24: mol_type = other DNA
ggattcaccttcgatgattatgcc

SEQ ID NO: 68 - protein, 8 aa, synthetic construct
GFTFDDYA

SEQ ID NO: 69 - DNA, 24 bp, synthetic construct
  source 1..24: mol_type = other DNA
ataagtggtgatggcggtagcaca

SEQ ID NO: 70 - protein, 8 aa, synthetic construct
ISGDGGST

SEQ ID NO: 71 - DNA, 57 bp, synthetic construct
  source 1..57: mol_type = other DNA
gcaaaagatctccgtaatacgatttttggagtggttattcccgatgcttttgatatc

SEQ ID NO: 72 - protein, 19 aa, synthetic construct
AKDLRNTIFGVVIPDAFDI

SEQ ID NO: 73 - DNA, 324 bp, synthetic construct
  source 1..324: mol_type = other DNA
gacatccagatgacccagtctccttccaccctgtctgcatctgtaggagacagagtcaccatcacttgccgggccagtcagagcattaggagctggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacaatataatagttattcgtacacttttggccaggggaccaagctggagatcaaacga

SEQ ID NO: 74 - protein, 108 aa, synthetic construct
DIQMTQSPSTLSASVGDRVTITCRASQSIRSWLAWYQQKPGKAPKLLIYKASSLESGVPSRFSGSGSGTEFTLTISSLQPDDFATYYCQQYNSYSYTFGQGTKLEIKR

SEQ ID NO: 75 - DNA, 18 bp, synthetic construct
  source 1..18: mol_type = other DNA
cagagcattaggagctgg

SEQ ID NO: 76 - protein, 6 aa, synthetic construct
QSIRSW

SEQ ID NO: 77-78 - skipped (2 entries)

SEQ ID NO: 79 - DNA, 27 bp, synthetic construct
  source 1..27: mol_type = other DNA
caacaatataatagttattcgtacact

SEQ ID NO: 80 - protein, 9 aa, synthetic construct
QQYNSYSYT

SEQ ID NO: 81 - DNA, 354 bp, synthetic construct
  source 1..354: mol_type = other DNA
caggtgcagctggtggagtcggggggaggcttggtcaagcctggagggtccctgagactctcctgtgcagcctctggattcactttcagtgactactacatgagctggatccgtcaggctccagggaaggggctggagtgggtttcatacattggtagtagtggtgtcaacatgtactacgcagactctgtgaagggccgattcaccatctccagggacaacgccaagaattcattatatctggaaatgaacagcctgagagccgaggacacggctgtttattactgtgcgagagactcttcccaactgggttttgactactggggccagggaaccctggtcaccgtctcctca

SEQ ID NO: 82 - protein, 118 aa, synthetic construct
QVQLVESGGGLVKPGGSLRLSCAASGFTFSDYYMSWIRQAPGKGLEWVSYIGSSGVNMYYADSVKGRFTISRDNAKNSLYLEMNSLRAEDTAVYYCARDSSQLGFDYWGQGTLVTVSS

SEQ ID NO: 83 - DNA, 24 bp, synthetic construct
  source 1..24: mol_type = other DNA
ggattcactttcagtgactactac

SEQ ID NO: 84 - protein, 8 aa, synthetic construct
GFTFSDYY

SEQ ID NO: 85 - DNA, 24 bp, synthetic construct
  source 1..24: mol_type = other DNA
attggtagtagtggtgtcaacatg

SEQ ID NO: 86 - protein, 8 aa, synthetic construct
IGSSGVNM

SEQ ID NO: 87 - DNA, 33 bp, synthetic construct
  source 1..33: mol_type = other DNA
gcgagagactcttcccaactgggttttgactac

SEQ ID NO: 88 - protein, 11 aa, synthetic construct
ARDSSQLGFDY

SEQ ID NO: 89 - DNA, 324 bp, synthetic construct
  source 1..324: mol_type = other DNA
gacatccagatgacccagtctccatcctccctgtctgcatctgtaggagacagagtcaccatcacttgccggacaagtcagaatattatcaactttttaaattggtatcaacagaaacctgggaaggcccctaaactcctgatctatactacttccactttacaaagtggggtcccatcaaggttcagtggcagtggatctgggacagatttcactctctccatcaatagtctacaacctgaagattttgcaacttacttctgtcaacagacttacagtaatccactcactttcggcggagggaccaaggtggagatcaaacga

SEQ ID NO: 90 - protein, 108 aa, synthetic construct
DIQMTQSPSSLSASVGDRVTITCRTSQNIINFLNWYQQKPGKAPKLLIYTTSTLQSGVPSRFSGSGSGTDFTLSINSLQPEDFATYFCQQTYSNPLTFGGGTKLEIKR

SEQ ID NO: 91 - DNA, 18 bp, synthetic construct
  source 1..18: mol_type = other DNA
cagaatattatcaacttt

SEQ ID NO: 92 - protein, 6 aa, synthetic construct
QNIINF

SEQ ID NO: 93-94 - skipped (2 entries)

SEQ ID NO: 95 - DNA, 27 bp, synthetic construct
  source 1..27: mol_type = other DNA
caacagacttacagtaatccactcact

SEQ ID NO: 96 - protein, 9 aa, synthetic construct
QQTYSNPLT

SEQ ID NO: 97 - DNA, 360 bp, synthetic construct
  source 1..360: mol_type = other DNA
gaggtgcagctggtgcagtctgggggaggcgtggtccagcctgggaggtccctgagactctcctgtgcagcgtctggattcaccttcagtaattatggcatgcactgggtccgccaggctccaggcaaggggctggagtgggtggcagttgtttggtatgatggagataataaatactatgcagactccgtgaagggccgattcaccatctccagagacaattccaagaacacgctgtatctgcaaatgaacagcctgagagccgaggacacggctgtgtattactgtgcgagagatattatcacatctcgcccgactttggactactggggccagggaaccctggtcactgtctcctca

SEQ ID NO: 98 - protein, 120 aa, synthetic construct
EVQLVQSGGGVVQPGRSLRLSCAASGFTFSNYGMHWVRQAPGKGLEWVAVVWYDGDNKYYADSVKGRFTISRDNSKNTLYLQMNSLRAEDTAVYYCARDIITSRPTLDYWGQGTLVTVSS

SEQ ID NO: 99 - DNA, 24 bp, synthetic construct
  source 1..24: mol_type = other DNA
ggattcaccttcagtaattatggc

SEQ ID NO: 100 - protein, 8 aa, synthetic construct
GFTFSNYG

SEQ ID NO: 101 - DNA, 24 bp, synthetic construct
  source 1..24: mol_type = other DNA
gtttggtatgatggagataataaa

SEQ ID NO: 102 - protein, 8 aa, synthetic construct
VWYDGDNK

SEQ ID NO: 103 - DNA, 39 bp, synthetic construct
  source 1..39: mol_type = other DNA
gcgagagatattatcacatctcgcccgactttggactac

SEQ ID NO: 104 - protein, 13 aa, synthetic construct
ARDIITSRPTLDY

SEQ ID NO: 105 - DNA, 324 bp, synthetic construct
  source 1..324: mol_type = other DNA
gacatccagttgacccagtctccatccttcctgtctgcatctgtaggagacagagtcaccatcacttgctgggccagtcagggcattaacagttatttagcctggtatcagcaaaaaccagggaaagcccctaagctcctgatctatcctgcatccactttgcaaagtggggtcccatcaaggttcagcggcagtggatctgggacagaattcactctcacaatcagcagcctgcagcctgtagattttgcaacttattactgtcaacagcttaatagttacccgctcactttcggcggagggaccaaggtggagatcaaacga

SEQ ID NO: 106 - protein, 108 aa, synthetic construct
DIQLTQSPSFLSASVGDRVTITCWASQGINSYLAWYQQKPGKAPKLLIYPASTLQSGVPSRFSGSGSGTEFTLTISSLQPVDFATYYCQQLNSYPLTFGGGTKVEIKR

SEQ ID NO: 107 - DNA, 18 bp, synthetic construct
  source 1..18: mol_type = other DNA
cagggcattaacagttat

SEQ ID NO: 108 - protein, 6 aa, synthetic construct
QGINSY

SEQ ID NO: 109-110 - skipped (2 entries)

SEQ ID NO: 111 - DNA, 27 bp, synthetic construct
  source 1..27: mol_type = other DNA
caacagcttaatagttacccgctcact

SEQ ID NO: 112 - protein, 9 aa, synthetic construct
QQLNSYPLT

SEQ ID NO: 113 - DNA, 354 bp, synthetic construct
  source 1..354: mol_type = other DNA
caggtgcagctggtggagtcggggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatatccagggacaacgccaagaactcactgtatctgcaaatgaacagcctgagagccgaggacacggccgtgtattactgtgcgagagattcttcccaactgggttttgactactggggccagggaaccctggtcaccgtctcctca

SEQ ID NO: 114 - protein, 118 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDSSQLGFDYWGQGTLVTVSS

SEQ ID NO: 115 - DNA, 24 bp, synthetic construct
  source 1..24: mol_type = other DNA
ggattcaccttcagtgactactac

SEQ ID NO: 116 - protein, 8 aa, synthetic construct
GFTFSDYY

SEQ ID NO: 117 - DNA, 24 bp, synthetic construct
  source 1..24: mol_type = other DNA
attagtagtagtggtagtaccata

SEQ ID NO: 118 - protein, 8 aa, synthetic construct
ISSSGSTI

SEQ ID NO: 119 - DNA, 33 bp, synthetic construct
  source 1..33: mol_type = other DNA
gcgagagattcttcccaactgggttttgactac

SEQ ID NO: 120 - protein, 11 aa, synthetic construct
ARDSSQLGFDY

SEQ ID NO: 121 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattatcagctttttaaattggtatcagcagaaaccagggaaggcccctaagctcctgatctatactgcatccagtttgcaaagtggggtcccatcaaggttcagtggcagtggatctgggacagatttcactctcaccatcagcagtctacaacctgaagattttgcaacttactactgtcaacagacttacagtaatccgctcactttcggcggagggaccaaggtggaaatcaaacga

SEQ ID NO: 122 - protein, 108 aa, synthetic construct
DIQMTQSPSSLSASVGDRVTITCRASQSIISFLNWYQQKPGKAPKLLIYTASSLQSGVPSRFSGSGSGTDFTLTISSLQPEDFATYYCQQTYSNPLTFGGGTKVEIKR

SEQ ID NO: 123 - DNA, 18 bp, synthetic construct
  source 1..18: mol_type = other DNA
cagagcattatcagcttt

SEQ ID NO: 124 - protein, 6 aa, synthetic construct
QSIISF

SEQ ID NO: 125-126 - skipped (2 entries)

SEQ ID NO: 127 - DNA, 27 bp, synthetic construct
  source 1..27: mol_type = other DNA
caacagacttacagtaatccgctcact

SEQ ID NO: 128 - protein, 9 aa, synthetic construct
QQTYSNPLT

SEQ ID NO: 129 - DNA, 342 bp, synthetic construct
  source 1..342: mol_type = other DNA
caggtgcagctggtacagtctggacctgaggtgaagaagcctggggcctcagtgagggtctcctgtaaggcttctggttaccttagtgactttattatcacctgggtgcgacaggcccctggacaagggcttgagtggatgggatggatcagcacttacagtggtgacacagactctgcaccgaagttccagggcagagtcaccatgaccacagacacatccacgactacagtcttcttggaactgaggagcctgagatctgacgacacggccgtgtattattgtgcgagagggcggctgtttgactactggggccagggaaccctggtcaccgtctcctca

SEQ ID NO: 130 - protein, 114 aa, synthetic construct
QVQLVQSGPEVKKPGASVRVSCKASGYLSDFIITWVRQAPGQGLEWMGWISTYSGDTDSAPKFQGRVTMTTDTSTTTVFLELRSLRSDDTAVYYCARGRLFDYWGQGTLVTVSS

SEQ ID NO: 131 - DNA, 21 bp, synthetic construct
  source 1..21: mol_type = other DNA
ggttaccttagtgactttatt

SEQ ID NO: 132 - protein, 7 aa, synthetic construct
GYLSDFI

SEQ ID NO: 133 - DNA, 24 bp, synthetic construct
  source 1..24: mol_type = other DNA
atcagcacttacagtggtgacaca

SEQ ID NO: 134 - protein, 8 aa, synthetic construct
ISTYSGDT

SEQ ID NO: 135 - DNA, 24 bp, synthetic construct
  source 1..24: mol_type = other DNA
gcgagagggcggctgtttgactac

SEQ ID NO: 136 - protein, 8 aa, synthetic construct
ARGRLFDY

SEQ ID NO: 137 - DNA, 339 bp, synthetic construct
  source 1..339: mol_type = other DNA
gatgttgtgatgactcagtctccactctccctgcccgtcacccttggacagccggcctccatctcctgcaggtctagtcaaagcctcgtatacagtgatggaaacacctacttgaattggtttcaacagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtacacttttggccaggggaccaagctggagatcaaacga

SEQ ID NO: 138 - protein, 113 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPYTFGQGTKLEIKR

SEQ ID NO: 139 - DNA, 33 bp, synthetic construct
  source 1..33: mol_type = other DNA
caaagcctcgtatacagtgatggaaacacctac

SEQ ID NO: 140 - protein, 11 aa, synthetic construct
QSLVYSDGNTY

SEQ ID NO: 141-142 - skipped (2 entries)

SEQ ID NO: 143 - DNA, 27 bp, synthetic construct
  source 1..27: mol_type = other DNA
atgcaaggtacacactggccgtacact

SEQ ID NO: 144 - protein, 9 aa, synthetic construct
MQGTHWPYT

SEQ ID NO: 145 - DNA, 354 bp, synthetic construct
  source 1..354: mol_type = other DNA
caggtgcagctggtggagtctgggggagtctcggtcaagcctggagggtccctgcgactctcctgtgcagcctctggattcaccttcagtgactactacatgagctggatccgccaggcgccagggaagggactggagtgggtttcgtacattggtagtagtggtactaatgactactacgcagactctgtgaagggccgattcaccatctccagggacaacgccaagaactcactgtatcttcaaatggacagcctgagagccgaggacacggccgtctattactgtgcgagagattcttcccaaatgggttttgactactggggccagggaaccctggtcactgtctcctca

SEQ ID NO: 146 - protein, 118 aa, synthetic construct
QVQLVESGGVSVKPGGSLRLSCAASGFTFSDYYMSWIRQAPGKGLEWVSYIGSSGTNDYYADSVKGRFTISRDNAKNSLYLQMDSLRAEDTAVYYCARDSSQMGFDYWGQGTLVTVSS

SEQ ID NO: 147 - DNA, 24 bp, synthetic construct
  source 1..24: mol_type = other DNA
ggattcaccttcagtgactactac

SEQ ID NO: 148 - protein, 8 aa, synthetic construct
GFTFSDYY

SEQ ID NO: 149 - DNA, 24 bp, synthetic construct
  source 1..24: mol_type = other DNA
attggtagtagtggtactaatgac

SEQ ID NO: 150 - protein, 8 aa, synthetic construct
IGSSGTND

SEQ ID NO: 151 - DNA, 33 bp, synthetic construct
  source 1..33: mol_type = other DNA
gcgagagattcttcccaaatgggttttgactac

SEQ ID NO: 152 - protein, 11 aa, synthetic construct
ARDSSQMGFDY

SEQ ID NO: 153 - DNA, 324 bp, synthetic construct
  source 1..324: mol_type = other DNA
gacatccagatgacccagtctccatcctccctgtctgcttctgtgggagacagagtcaccatcacttgccgggcaagtcagaacattatcaactttttaaattggtatcagcagagaccagggaaagcccctcagctcctgatctatgttgcagccagcttgcagagtggggtcccatcaaggttcactggcagtggatatgggacagatttcactctcaccatcagcagtctgcaacctgaggatttcgcaacttactactgtcaacagacttacactaacccgctcactttcggcggagggaccaaggtggagatcaaacga

SEQ ID NO: 154 - protein, 108 aa, synthetic construct
DIQMTQSPSSLSASVGDRVTITCRASQNIINFLNWYQQRPGKAPQLLIYVAASLQSGVPSRFTGSGYGTDFTLTISSLQPEDFATYYCQQTYTNPLTFGGGTKVEIKR

SEQ ID NO: 155 - DNA, 18 bp, synthetic construct
  source 1..18: mol_type = other DNA
cagaacattatcaacttt

SEQ ID NO: 156 - protein, 6 aa, synthetic construct
QNIINF

SEQ ID NO: 157-158 - skipped (2 entries)

SEQ ID NO: 159 - DNA, 27 bp, synthetic construct
  source 1..27: mol_type = other DNA
caacagacttacactaacccgctcact

SEQ ID NO: 160 - protein, 9 aa, synthetic construct
QQTYTNPLT

SEQ ID NO: 161 - protein, 460 aa, Homo sapiens
MFTIKLLLFIVPLVISSRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEKQDNSIKDLLQTVEDQYKQLNQQHSQIKEIENQLRRTSIQEPTEISLSSKPRAPRTTPFLQLNEIRNVKHDGIPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAKSKPERRRGLSWKSQNGRLYSIKSTKMLIHPTDSESFE

SEQ ID NO: 162 - DNA, 1383 bp, Homo sapiens
  source 1..1383: mol_type = genomic DNA
atgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaatga

SEQ ID NO: 163 - protein, 455 aa, Mus musculus
MHTIKLFLFVVPLVIASRVDPDLSSFDSAPSEPKSRFAMLDDVKILANGLLQLGHGLKDFVHKTKGQINDIFQKLNIFDQSFYDLSLRTNEIKEEEKELRRTTSTLQVKNEEVKNMSVELNSKLESLLEEKTALQHKVRALEEQLTNLILSPAGAQEHPEVTSLKSFVEQQDNSIRELLQSVEEQYKQLSQQHMQIKEIEKQLRKTGIQEPSENSLSSKSRAPRTTPPLQLNETENTEQDDLPADCSAVYNRGEHTSGVYTIKPRNSQGFNVYCDTQSGSPWTLIQHRKDGSQDFNETWENYEKGFGRLDGEFWLGLEKIYAIVQQSNYILRLELQDWKDSKHYVEYSFHLGSHETNYTLHVAEIAGNIPGALPEHTDLMFSTWNHRAKGQLYCPESYSGGWWWNDICGENNLNGKYNKPRTKSRPERRRGIYWRPQSRKLYAIKSSKMMLQPTT

SEQ ID NO: 164 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 165 - protein, 386 aa, synthetic construct
SRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 166 - protein, 235 aa, synthetic construct
GAPSRVDPDLSSFDSAPSEPKSRFAMLDDVKILANGLLQLGHGLKDFVHKTKGQINDIFQKLNIFDQSFYDLSLRTNEIKEEEKELRRTTSTLQVKNEEVKNMSVELNSKLESLLEEKTALQHKVRALEEQLTNLILSPAGAQEHPEVTSLKSFVEQQDNSIRELLQSVEEQYKQLSQQHMQIKEIEKQLRKTGIQEPSENSLSSKSRAPRTTPPLQLNETENTEQDASHHHHHH

SEQ ID NO: 167 - protein, 182 aa, synthetic construct
SRIDQDNSSFDSVSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPATPEHPEVTSLKSFVEKEQKLISEEDLGGEQKLISEEDLHHHHHH

SEQ ID NO: 168 - protein, 33 aa, synthetic construct
RFASWDEMNVLAHGLLQLGQGLREHAERTRSQL

SEQ ID NO: 169 - protein, 33 aa, synthetic construct
RFAMLDDVKILANGLLQLGHGLKDFVHKTKGQI

SEQ ID NO: 170 - protein, 26 aa, synthetic construct
EPKSRFAMLDDVKILANGLLQLGHGL

SEQ ID NO: 171 - protein, 7 aa, synthetic construct
AGSSPGG

SEQ ID NO: 172 - protein, 5 aa, synthetic construct
GGGGS

SEQ ID NO: 173 - protein, 10 aa, synthetic construct
GPSSGAPPPK

SEQ ID NO: 174 - protein, 6 aa, synthetic construct
GGGGSK

SEQ ID NO: 175 - protein, 455 aa, Rattus norvegicus
MHTIKLLLFVVPLVISSRVDPDLSPFDSVPSEPKSRFAMLDDVKILANGLLQLGHGLKDFVHKTKGQINDIFQKLNIFDQCFYDLSLQTNEIKEEEKELRRTTSKLQVKNEEVKNMSLELNSKLESLLEEKMALQHRVRALEEQLTSLVQNPPGAREHPEVTSLKSFVEQQDNSIRELLQSVEEQYKQLSQQHIQIKEIENQLRKTGIQEPTENSLYSKPRAPRTTPPLHLKEAKNIEQDDLPADCSAIYNRGEHTSGVYTIRPSSSQVFNVYCDTQSGTPRTLIQHRKDGSQNFNQTWENYEKGFGRLDGEFWLGLEKIYAIVKQSNYILRLELQDWKDSKHYAEYSFHLGNHETNYTLHVAEIAANIPEALPEHRDLMFSTWDHRAKGQLYCPESYSGGWWFSDMCGENNLNGKYNKPRAKSKPERRRGISWRPRGGKLYSIKSSKMMLQPTT

SEQ ID NO: 176 - protein, 252 aa, synthetic construct
SRVDPDLSPFDSVPSEPKSRFAMLDDVKILANGLLQLGHGLKDFVHKTKGQINDIFQKLNIFDQCFYDLSLQTNEIKEEEKELRRTTSKLQVKNEEVKNMSLELNSKLESLLEEKMALQHRVRALEEQLTSLVQNPPGAREHPEVTSLKSFVEQQDNSIRELLQSVEEQYKQLSQQHIQIKEIENQLRKTGIQEPTENSLYSKPRAPRTTPPLHLKEAKNIEQDEQKLISEEDLGGEQKLISEEDLHHHHHH

SEQ ID NO: 177 - protein, 210 aa, Macaca fascicularis
MFTIKLLLFIVPLVISSRIDQDNSSFDSVSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPATPEHPEVTSLKSFVEKQDNSIKDLLQTVEEQYKQLNQQHSQIKEIENQLRMTNIQE

SEQ ID NO: 178 - protein, 356 aa, synthetic construct
GPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 179 - DNA, 363 bp, synthetic construct
  source 1..363: mol_type = other DNA
gaggtgcagcttttggagtctgggggaggcttggtacagcctggggggtccctgagactctcctgtgcagcctctggattcacctttagcacctatgccatgagctgggtccgccaggctccagggaaggggctggagggggtctcaggtattagtggtactggttatagaacatactacgcagactccgtgaagggccgattcaccatctccagagacaattccaagaactcgctgtatctgcaaatgaacagcctgagagccgaggacacggccgtatattactgtgcgaaagatcggggcttactatggttcggggaattaacctactggggccagggaaccctggtcaccgtctcctca

SEQ ID NO: 180 - protein, 121 aa, synthetic construct
EVQLLESGGGLVQPGGSLRLSCAASGFTFSTYAMSWVRQAPGKGLEGVSGISGTGYRTYYADSVKGRFTISRDNSKNSLYLQMNSLRAEDTAVYYCAKDRGLLWFGELTYWGQGTLVTVSS

SEQ ID NO: 181 - DNA, 24 bp, synthetic construct
  source 1..24: mol_type = other DNA
ggattcacctttagcacctatgcc

SEQ ID NO: 182 - protein, 8 aa, synthetic construct
GFTFSTYA

SEQ ID NO: 183 - DNA, 24 bp, synthetic construct
  source 1..24: mol_type = other DNA
attagtggtactggttatagaaca

SEQ ID NO: 184 - protein, 8 aa, synthetic construct
ISGTGYRT

SEQ ID NO: 185 - DNA, 42 bp, synthetic construct
  source 1..42: mol_type = other DNA
gcgaaagatcggggcttactatggttcggggaattaacctac

SEQ ID NO: 186 - protein, 14 aa, synthetic construct
AKDRGLLWFGELTY

SEQ ID NO: 187 - DNA, 318 bp, synthetic construct
  source 1..318: mol_type = other DNA
gacatccagatgacccagtctccttccaccctgtctgcatctgtaggagacagagtcaccatcacttgccgggccagtcagagtattaataactggttggcctggtatcaacagaaaccagggaaggcccctaacctcctgatctataaggcgtctagtttagaaagtggggtcccatcaaggttcagcggcagtggatctgggacagaattcactctcaccatcagcagcctgcagcctgatgattttgcaacttattactgccaacaatataatgattattggacgttcggccaagggaccaaggtggaaatcaaa

SEQ ID NO: 188 - protein, 106 aa, synthetic construct
DIQMTQSPSTLSASVGDRVTITCRASQSINNWLAWYQQKPGKAPNLLIYKASSLESGVPSRFSGSGSGTEFTLTISSLQPDDFATYYCQQYNDYWTFGQGTKVEIK

SEQ ID NO: 189 - DNA, 18 bp, synthetic construct
  source 1..18: mol_type = other DNA
cagagtattaataactgg

SEQ ID NO: 190 - protein, 6 aa, synthetic construct
QSINNW

SEQ ID NO: 191-192 - skipped (2 entries)

SEQ ID NO: 193 - DNA, 24 bp, synthetic construct
  source 1..24: mol_type = other DNA
caacaatataatgattattggacg

SEQ ID NO: 194 - protein, 8 aa, synthetic construct
QQYNDYWT

SEQ ID NO: 195 - protein, 6 aa, synthetic construct
HHHHHH

SEQ ID NO: 196 - protein, 10 aa, synthetic construct
HHHHHHHHHH

(19) United States
(12) Patent Application Publication
(10) Pub. No.: US 2025/0136675 A1
(43) Pub. Date: May 1, 2025

(54) ANTI-ANGPTL3 ANTIBODIES AND USES THEREOF

(71) Applicant: REGENERON PHARMACEUTICALS, INC., Tarrytown, NY (US)

(72) Inventors: Mark W. SLEEMAN, New York, NY (US); Viktoria GUSAROVA, Pleasantville, NY (US); Andrew J. MURPHY, Croton-on-Hudson, NY (US)

(21) Appl. No.: 19/004,070

(22) Filed: Dec. 27, 2024

Related U.S. Application Data
(63) Continuation of application No. 17/930,704, filed on Sep. 8, 2022.
Application No. 17/930,704 is a continuation of application No. 17/586,662, filed on Jan. 27, 2022, now abandoned.
Application No. 17/586,662 is a continuation of application No. 16/435,032, filed on Jun. 7, 2019, now abandoned.
Application No. 16/435,032 is a continuation of application No. 15/958,996, filed on Apr. 20, 2018, now Pat. No. 10,358,487.
Application No. 15/958,996 is a division of application No. 14/667,121, filed on Mar. 24, 2015, now Pat. No. 9,951,127.
Application No. 14/667,121 is a division of application No. 13/517,662, filed on Jun. 14, 2012, now Pat. No. 9,018,356.
(60) Provisional application No. 61/578,309, filed on Dec. 21, 2011.
(60) Provisional application No. 61/498,518, filed on Jun. 17, 2011.

(51) Int. Cl. C07K 16/22; A61K 31/455; A61K 39/00; A61K 39/395; A61K 45/06

(52) U.S. Cl. CPC C07K 16/22; A61K 31/455; A61K 39/3955; A61K 45/06; A61K 2039/505; C07K 2317/21; C07K 2317/33; C07K 2317/34; C07K 2317/76; C07K 2317/92

(57) ABSTRACT

A fully human antibody or antigen-binding fragment of a human antibody that specifically binds and inhibits or interferes with at least one activity of human angiopoietin-like protein 3 (hANGPTL3) is provided. The human anti-hANGPTL3 antibodies are useful in treating diseases or disorders associated with ANGPTL3, such as hyperlipidemia, hyperlipoproteinemia and dyslipidemia, including hypertriglyceridemia, hypercholesterolemia, chylomicronemia, and so forth. Furthermore, the anti-hANGPTL3 antibodies can be administered to a subject in need thereof to prevent or treat diseases or disorders, for which abnormal lipid metabolism is a risk factor. Such diseases or disorders include cardiovascular diseases, such as atherosclerosis and coronary artery diseases; acute pancreatitis; nonalcoholic steatohepatitis (NASH); diabetes; obesity; and the like.

### CROSS-REFERENCE TO RELATED APPLICATIONS

This application is a continuation of U.S. patent application Ser. No. 17/930,704, filed Sep. 8, 2022, entitled, “Anti-ANGPTL3 Antibodies and Uses Thereof,” which is a continuation of U.S. patent application Ser. No. 17/586,662, filed Jan. 27, 2022, entitled, “Anti-ANGPTL3 Antibodies and Uses Thereof,” now abandoned, which is a continuation of U.S. patent application Ser. No. 16/435,032, filed Jun. 7, 2019, entitled, “Anti-ANGPTL3 Antibodies and Uses Thereof,” now abandoned, which is a continuation of U.S. patent application Ser. No. 15/958,996, filed Apr. 20, 2018, now issued as U.S. Pat. No. 10,358,487, entitled “Anti-ANGPTL3 Antibodies and Uses Thereof,” which is a divisional of U.S. patent application Ser. No. 14/667,121 filed Mar. 24, 2015, now issued as U.S. Pat. No. 9,951,127, entitled “Anti-ANGPTL3 Antibodies and Uses Thereof,” which is a divisional of U.S. patent application Ser. No. 13/517,662 filed Jun. 14, 2012, now issued as U.S. Pat. No. 9,018,356, entitled “Anti-ANGPTL3 Antibodies and Uses Thereof,” which claims the benefit under 35 U.S.C. § 119(e) of U.S. provisional application Nos. 61/498,518, filed on Jun. 17, 2011; and 61/578,309, filed on Dec. 21, 2011, the disclosures of which are herein incorporated by reference in their entireties.

### REFERENCE TO SEQUENCE LISTING XML

The instant application contains a Sequence Listing, which has been submitted electronically in XML format and is hereby incorporated by reference in its entirety. Said XML file, created on Sep. 8, 2022, is named 40848_0079USC3_SL.xml and is 172,032 bytes in size.

### FIELD OF THE INVENTION

The present invention is related to human antibodies and antigen-binding fragments of human antibodies that specifically bind human angiopoietin-like protein 3 (hANGPTL3), and therapeutic methods of using those antibodies.

### BACKGROUND

The angiopoietin-like protein 3 (ANGPTL3) gene was identified from the EST database based on signal sequences and amphipathic helices, and a full-length ANGPTL3 cDNA was subsequently isolated from a human fetal liver/spleen cDNA library (Conklin, et al., 1999, Genomics62:477-482). The deduced 460-amino acid hANGPTL3 protein shares 76% amino acid sequence identity with mouse ANGPTL3 and has the characteristic structure of angiopoietins; i.e., a signal peptide, an extended helical domain predicted to form dimeric or trimeric coiled-coils, a short linker peptide, and a globular fibrinogen homology domain (FD) (Conklin, et al., 1999, supra). ANGPTL3 contains the 4 conserved cysteine residues implicated in the intramolecular disulfide bonds within the FD; however, ANGPTL3 contains neither the two additional cysteines nor the characteristic calcium-binding motif found in the FDs of angiopoietins (ANGs; i.e., ANG1, ANG2 and ANG4) (Conklin, et al., 1999, supra), which are protein growth factors that promote angiogenesis. In addition, unlike ANGs, ANGPTL3 does not bind to Tie2; however, it may also induce angiogenesis by binding to integrin αᵥβ₃ via its C-terminal FD (Camenisch, et al., 2002, J Biol Chem277:17281-17290).

Comprehensive in vivo data were obtained from the outbred KK mouse model, which is moderately obese with abnormally high levels of plasma insulin, glucose, and lipids, resembling type 2 diabetes mellitus in humans (Koishi, et al., 2002, Nature Genetics30:151-157). One sub-strain of mouse, the KK/San, however, was found to exhibit abnormally low plasma lipid levels (hypolipidemia), which were inherited as a Mendelian recessive. The loci was mapped to chromosome 4 and eventually identified to be the gene encoding ANGPTL3, which contained a 4-bp nucleotide sequence insertion in exon 6 (Koishi et al., 2002, supra). Conversely, plasma lipid levels increase after adenovirus-mediated transfer of ANGPTL3 gene, or after administration of recombinant human ANGPTL3 in KK/San mice. This effect was not mediated by changes in genes involved in cholesterol synthesis, lipoprotein clearance or NEFA oxidation (Koishi, et al., 2002, supra). Further, in vitro analysis of recombinant protein showed that ANGPTL3 directly inhibits lipoprotein lipase (LPL) activity, indicating that it is a lipid metabolism modulator that regulates very low density lipoprotein (VLDL) triglyceride levels through the inhibition of LPL activity (Shimizugawa, et al., 2002, J Biol Chem 277(37):33742-33748). It has been shown that the N-terminal coiled-coil domain, especially the N-terminal region residues 17-165, and not the C-terminal FD, of ANGPTL3, is required for its activity of increasing plasma triglyceride levels in mice (Ono, et al., 2003, J Biol Chem 278:41804-41809).

The amino acid and nucleotide sequences of human ANGPTL3 are shown in SEQ ID NOS: 161 and 162, respectively. Antibodies to ANGPTL3 are disclosed in, for example, WO2008/073300 and U.S. Pat. No. 7,935,796.

### BRIEF SUMMARY OF THE INVENTION

In a first aspect, the invention provides fully human monoclonal antibodies (mAbs) and antigen-binding fragments thereof that specifically bind and neutralize, inhibit, block, abrogate, reduce or interfere with, at least one activity of ANGTPL3, in particular, human ANGPTL3 (SEQ ID NO:161). The activity of ANGPTL3 that can be neutralized, inhibited, blocked, abrogated, reduced or interfered with, by the antibodies or fragments thereof of the invention, includes, but not by the way of limitation, inhibition of LPL activity, induction of angiogenesis, and the like. In one embodiment, an antibody or fragment thereof of the present invention can neutralize, inhibit, block, abrogate, reduce or interfere with, an activity of hANGPTL3 by binding to an epitope of hANGPTL3 that is directly involved in the targeted activity of hANGPTL3. In another embodiment, an antibody or fragment thereof of the invention can neutralize, inhibit, block, abrogate, reduce or interfere with, an activity of hANGPTL3 by binding to an epitope of hANGPTL3 that is not directly involved in the targeted activity of hANGPTL3, but the antibody or fragment binding thereto sterically or conformationally inhibits, blocks, abrogates, reduces or interferes with, the targeted activity of hANGPTL3. In yet another embodiment, an antibody or fragment thereof of the invention binds to an epitope of hANGPTL3 that is not directly involved in the targeted activity (e.g., inhibiting LPL activity, inducing angiogenesis, and the like) of hANGPTL3 (i.e., a non-blocking antibody), but the antibody or fragment binding thereto results in the enhancement of the clearance of hANGPTL3 from the circulation, compared to the clearance of hANGPTL3 in the absence of the antibody or fragment thereof, thereby indirectly inhibiting, blocking, abrogating, reducing or interfering with, an activity of hANGPTL3. Clearance of hANGPTL3 from the circulation can be particularly enhanced by combining two or more different non-blocking antibodies that do not compete with one another for specific binding to hANGPTL3.

The antibodies (Abs) can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy, et al., 2000, J. Immunol. 164:1925-1933).

In one embodiment, the invention comprises an antibody or antigen-binding fragment of an antibody comprising a heavy chain variable region (HCVR) selected from the group consisting of SEQ ID NO:2, 18, 34, 50, 66, 82, 98, 114, 130, 146 and 180, or a substantially similar sequence thereof having at least 90%, at least 95%, at least 98% or at least 99% sequence identity. In another embodiment, the antibody or antigen-binding fragment thereof comprises a HCVR having an amino acid sequence selected from the group consisting of SEQ ID NO:2, 18, 34, 66, 82, 114, and 180. In yet another embodiment, the antibody or an antigen-binding fragment thereof comprises a HCVR having an amino acid sequence of SEQ ID NO:66.

In one embodiment, an antibody or antigen-binding fragment of an antibody comprises a light chain variable region (LCVR) selected from the group consisting of SEQ ID NO:10, 26, 42, 58, 74, 90, 106, 122, 138, 154 and 188, or a substantially similar sequence thereof having at least 90%, at least 95%, at least 98% or at least 99% sequence identity. In another embodiment, the antibody or antigen-binding portion of an antibody comprises a LCVR having an amino acid sequence selected from the group consisting of SEQ ID NO:10, 26, 42, 74, 90, 122 and 188. In yet another embodiment, the antibody or antigen-binding portion of an antibody comprises a LCVR having an amino acid sequence of SEQ ID NO: 74.

In further embodiments, the antibody or fragment thereof comprises a HCVR and LCVR sequence pair (HCVR/LCVR) selected from the group consisting of SEQ ID NO:2/10, 18/26, 34/42, 50/58, 66/74, 82/90, 98/106, 114/122, 130/138, 146/154 and 180/188. In one embodiment, the antibody or fragment thereof comprises a HCVR and LCVR sequence pair selected from the group consisting of SEQ ID NO:2/10, 18/26, 34/42, 66/74, 82/90, 114/122 and 180/188. In another embodiment, the antibody or fragment thereof comprises a HCVR and LCVR sequence pair of SEQ ID NO:66/74.

In a second aspect, the invention features an antibody or antigen-binding fragment of an antibody comprising a heavy chain complementarity determining region 3 (HCDR3) amino acid sequence selected from the group consisting of SEQ ID NO:8, 24, 40, 56, 72, 88, 104, 120, 136, 152 and 186, or a substantially similar sequence thereof having at least 90%, at least 95%, at least 98% or at least 99% sequence identity; and a light chain CDR3 (LCDR3) amino acid sequence selected from the group consisting of SEQ ID NO:16, 32, 48, 64, 80, 96, 112, 128, 144, 160 and 194, or substantially similar sequences thereof having at least 90%, at least 95%, at least 98% or at least 99% sequence identity. In one embodiment, the antibody or fragment thereof comprises a HCDR3/LCDR3 amino acid sequence pair comprising SEQ ID NO:8/16, 24/32, 40/48, 56/64, 72/80, 88/96, 104/112, 120/128, 136/144, 152/160 or 186/194. In another embodiment, the antibody or fragment thereof comprises a HCDR3/LCDR3 amino acid sequence pair comprising SEQ ID NO:8/16, 24/32, 40/48, 72/80, 88/96, 120/128 or 186/194. In yet another embodiment, the antibody or fragment thereof comprises a HCDR3/LCDR3 amino acid sequence pair comprising SEQ ID NO:72/80.

In a further embodiment, the antibody or fragment thereof further comprises a heavy chain CDR1 (HCDR1) amino acid sequence selected from the group consisting of SEQ ID NO:4, 20, 36, 52, 68, 84, 100, 116, 132, 148 and 182, or a substantially similar sequence thereof having at least 90%, at least 95%, at least 98% or at least 99% sequence identity; and a heavy chain CDR2 (HCDR2) amino acid sequence selected from the group consisting of SEQ ID NO:6, 22, 38, 54, 70, 86, 102, 118, 134, 150 and 184, or a substantially similar sequence thereof having at least 90%, at least 95%, at least 98% or at least 99% sequence identity; and optionally further comprises a light chain CDR1 (LCDR1) amino acid sequence selected from the group consisting of SEQ ID NO:12, 28, 44, 60, 76, 92, 108, 124, 140, 156 and 190, or a substantially similar sequence thereof having at least 90%, at least 95%, at least 98% or at least 99% sequence identity; and/or a light chain CDR2 (LCDR2) amino acid sequence selected from the group consisting of SEQ ID NO:AAS, KAS, AAS, KAS, KAS, TTS, PAS, TAS, KVS, VAA and KAS, or a substantially similar sequence thereof having at least 90%, at least 95%, at least 98% or at least 99% sequence identity.

Alternatively, the invention features an antibody or antigen-binding fragment of an antibody comprising a HCDR1/HCDR2/HCDR3 combination selected from the group consisting of SEQ ID NO:4/6/8, 20/22/24, 36/38/40, 52/54/56, 68/70/72, 84/86/88, 100/102/104, 116/118/120, 132/134/136, 148/150/152 and 182/184/186; and/or a LCDR1/LCDR2/LCDR3 combination selected from the group consisting of SEQ ID NO:12/AAS/16, 28/KAS/32, 44/AAS/48, 60/KAS/64, 76/KAS/80, 92/TTS/96, 108/PAS/112, 124/TAS/128, 140/KVS/144, 156/VAA/160 and 190/KAS/194. In one embodiment, the heavy and light chain CDR amino acid sequences comprise a CDR sequence combination selected from the group consisting of SEQ ID NO:4/6/8/12/AAS/16, 20/22/24/28/KAS/32, 36/38/40/44/AAS/48, 52/54/56/60/KAS/64, 68/70/72/76/KAS/80, 84/86/88/92/TTS/96, 100/102/104/108/PAS/112, 116/118/120/124/TAS/128, 132/134/136/140/KVS/144, 148/150/152/156/VAA/160 and 182/184/186/190/KAS/194. In one embodiment, the heavy and light chain CDR amino acid sequences comprise a CDR sequence combination of SEQ ID NO: 4/6/8/12/AAS/16, 20/22/24/28/KAS/32, 36/38/40/44/AAS/48 68/70/72/76/KAS/80, 84/86/88/92/TTS/96, 116/118/120/124/TAS/128 or 182/184/186/190/KAS/194. In another embodiment, the heavy and light chain CDR amino acid sequences comprise a CDR sequence combination of SEQ ID NO:68/70/72/76/KAS/80.

In a related embodiment, the invention comprises an antibody or antigen-binding fragment of an antibody which specifically binds hANGPTL3, wherein the antibody or fragment thereof comprises heavy and light chain CDR domains contained within HCVR/LCVR pairs selected from the group consisting of SEQ ID NO:2/10, 18/26, 34/42, 50/58, 66/74, 82/90, 98/106, 114/122, 130/138, 146/154 and 180/188. Methods and techniques for identifying CDRs within HCVR and LCVR amino acid sequences are known in the art and can be applied to identify CDRs within the specified HCVR and/or LCVR amino acid sequences disclosed herein. Conventional definitions that can be applied to identify the boundaries of CDRs include the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani, et al., J. Mol. Biol. 273:927-948 (1997); and Martin, et al., Proc. Natl. Acad. Sci. USA 86:9268-9272 (1989). Public databases are also available for identifying CDR sequences within an antibody. In one embodiment, the antibody or fragment thereof comprises CDR sequences contained within a HCVR and LCVR pair of SEQ ID NO: 2/10, 18/26, 34/42, 66/74, 82/90, 114/122 or 180/188. In another embodiment, the antibody or fragment thereof comprises CDR sequences contained within a HCVR and LCVR pair of SEQ ID NO:66/74.

In another related embodiment, the invention provides an antibody or antigen-binding fragment thereof that competes for specific binding to hANGPTL3 with an antibody or antigen-binding fragment comprising heavy and light chain CDR sequences contained in a HCVR/LCVR sequence pair of SEQ ID NO:2/10, 18/26, 34/42, 50/58, 66/74, 82/90, 98/106, 114/122, 130/138, 146/154 or 180/188. In one embodiment, the antibody or antigen-binding fragment of the invention competes for specific binding to hANGPTL3 with an antibody or fragment thereof comprising a HCVR/LCVR sequence pair of SEQ ID NO:66/74. In another embodiment, the antibody or antigen-binding fragment of the invention competes for specific binding to hANGPTL3 with an antibody or fragment thereof comprising a heavy and light chain CDR sequence combination selected from the group consisting of SEQ ID NOS 4/6/8/12/AAS/16, 20/22/24/28/KAS/32, 36/38/40/44/AAS/48, 52/54/56/60/KAS/64, 68/70/72/76/KAS/80, 84/86/88/92/TTS/96, 100/102/104/108/PAS/112, 116/118/120/124/TAS/128, 132/134/136/140/KVS/144, 148/150/152/156/VAA/160 and 182/184/186/190/KAS/194. In one embodiment, the antibody or antigen-binding fragment thereof of the invention competes for specific binding to hANGPTL3 with an antibody or fragment thereof comprising a heavy and light chain CDR sequence combination of SEQ ID NOS:68/70/72/76/KAS/80.

In another related embodiment, the invention provides an antibody or antigen-binding fragment thereof that binds the same epitope on hANGPTL3 that is recognized by an antibody or fragment thereof comprising heavy and light chain CDR sequences from a HCVR/LCVR sequence pair of SEQ ID NO:2/10, 18/26, 34/42, 50/58, 66/74, 82/90, 98/106, 114/122, 130/138, 146/154 or 180/188. In one embodiment, the antibody or antigen-biding fragment of the invention binds the same epitope on hANGPTL3 as that recognized by the antibody or fragment thereof comprising a HCVR/LCVR sequence pair of SEQ ID NO:66/74. In one embodiment, the antibody or fragment thereof of the invention binds the same epitope on hANGPTL3 that is recognized by an antibody or fragment thereof comprising a heavy and light chain CDR sequence combination selected from the group consisting of SEQ ID NOS 4/6/8/12/AAS/16, 20/22/24/28/KAS/32, 36/38/40/44/AAS/48, 52/54/56/60/KAS/64, 68/70/72/76/KAS/80, 84/86/88/92/TTS/96, 100/102/104/108/PAS/112, 116/118/120/124/TAS/128, 132/134/136/140/KVS/144, 148/150/152/156/VAA/160 and 182/184/186/190/KAS/194. In one embodiment, such an epitope is recognized by an antibody or fragment thereof comprising a heavy and light chain CDR sequence combination of SEQ ID NO:68/70/72/76/KAS/80.

In a third aspect, the invention features an isolated anti-hANGPTL3 antibody or antigen-binding fragment thereof that binds to an epitope situated within the N-terminal coiled-coil region at residues 17 to 209 of SEQ ID NO:161 and neutralizes, inhibits, abrogates, reduces or interferes with, at least one activity of hANGPTL3. In another embodiment, the invention provides an isolated antibody or antigen-binding fragment of an antibody that specifically binds to an epitope situated within the N-terminal coiled-coil region of hANGPTL3 (SEQ ID NO:161) and neutralizes, inhibits, abrogates, reduces or interferes with, at least one activity of hANGPTL3, with the proviso that the antibody or fragment thereof does not bind to the ANGPTL3 peptide of SEQ ID NO:170 (corresponds to residues Glu32 to Leu57 of hANGPTL3 of SEQ ID NO:161). In one embodiment, the antibody or fragment thereof of the invention specifically binds to an epitope within residues 17 to 200, 17 to 100, 17 to 70, 17 to 65, 17 to 60, 17 to 57, or 17 to 50, of hANGPTL3 (SEQ ID NO:161), optionally with the proviso that the antibody or fragment thereof does not bind to the ANGPTL3 peptide of SEQ ID NO:170. In another embodiment, the antibody or fragment thereof specifically binds to an epitope within residues 40 to 200, 40 to 100, 40 to 70, 50 to 200, 50 to 100, 50 to 70, 58 to 200, 58 to 100, 58 to 70, 58 to 68, or 61 to 66, of hANGPTL3 (SEQ ID NO:161), optionally with the proviso that the antibody or fragment thereof does not bind to the ANGPTL3 peptide of SEQ ID NO:170. In some embodiments, the antibody or antibody fragment binds an epitope which may involve more than one of the enumerated epitopes or residues within the N-terminal coiled-coil region of hANGPTL3, optionally with the proviso that the antibody or fragment thereof does not bind to the ANGPTL3 peptide of SEQ ID NO:170.

In a fourth aspect, the invention provides nucleic acid molecules encoding anti-ANGPTL3 antibodies or fragments thereof, in particular, any one of those described above. Recombinant expression vectors carrying the nucleic acids of the invention, and host cells, e.g., bacterial cells, such as E. coli, or mammalian cells, such as CHO cells, into which such vectors have been introduced, are also encompassed by the invention, as are methods of producing the antibodies by culturing the host cells under conditions permitting production of the antibodies, and recovering the antibodies produced.

In one embodiment, the invention provides an antibody or fragment thereof comprising a HCVR encoded by a nucleic acid sequence selected from the group consisting of SEQ ID NO: 1, 17, 33, 49, 65, 81, 97, 113, 129, 145 and 179, or a substantially identical sequence having at least 90%, at least 95%, at least 98%, or at least 99% homology thereof. In another embodiment, the antibody or fragment thereof comprises a HCVR encoded by a nucleic acid sequence of SEQ ID NO:1, 17, 33, 65, 81, 113 or 179. In yet another embodiment, the antibody or fragment thereof comprises a HCVR encoded by a nucleic acid sequence of SEQ ID NO:65.

In one embodiment, an antibody or antigen-binding fragment thereof comprises a LCVR encoded by a nucleic acid sequence selected from the group consisting of SEQ ID NO: 9, 25, 41, 57, 73, 89, 105, 121, 137, 153 and 187, or a substantially identical sequence having at least 90%, at least 95%, at least 98%, or at least 99% homology thereof. In another embodiment, the antibody or fragment thereof comprises a LCVR encoded by a nucleic acid sequence of SEQ ID NO:9, 25, 41, 73, 89, 121 or 187. In yet another embodiment, the antibody or fragment thereof comprises a LCVR encoded by a nucleic acid sequence of SEQ ID NO:73.

In further embodiments, the antibody or fragment thereof comprises a HCVR and LCVR (HCVR/LCVR) sequence pair encoded by a nucleic acid sequence pair selected from the group consisting of SEQ ID NO:1/9, 17/25, 33/41, 49/57, 65/73, 81/89, 97/105, 113/121, 129/137, 145/153 and 179/187. In one embodiment, the antibody or fragment thereof comprises a HCVR/LCVR sequence pair encoded by a nucleic acid sequence pair of SEQ ID NO:1/9, 17/25, 33/41, 65/73, 81/89, 113/121 or 179/187. In another embodiment, the antibody or fragment thereof comprises a HCVR/LCVR sequence pair encoded by a nucleic acid sequence pair of SEQ ID NO:65/73.

In one embodiment, the invention features an antibody or antigen-binding fragment of an antibody comprising a HCDR3 domain encoded by a nucleotide sequence selected from the group consisting of SEQ ID NO:7, 23, 39, 55, 71, 87, 103, 119, 135, 151 and 185, or a substantially identical sequence having at least 90%, at least 95%, at least 98%, or at least 99% homology thereof; and a LCDR3 domain encoded by a nucleotide sequence selected from the group consisting of SEQ ID NO:15, 31, 47, 63, 79, 95, 111, 127, 143, 159 and 193, or a substantially identical sequence having at least 90%, at least 95%, at least 98%, or at least 99% homology thereof. In one embodiment, the antibody or fragment thereof comprises a HCDR3 and LCDR3 sequence pair encoded by the nucleic acid sequence pair selected from the group consisting of SEQ ID NO:7/15, 23/31, 39/47, 55/63, 71/79, 87/95, 103/111, 119/127, 135/143, 151/159 and 185/193. In another embodiment, the antibody or fragment thereof comprises a HCDR3 and LCDR3 sequence pair encoded by the nucleic acid sequence pair of SEQ ID NO:7/15, 23/31, 39/47, 71/79, 87/95, 119/127 or 185/193. In yet another embodiment, the antibody or fragment thereof comprises a HCDR3 and LCDR3 sequence pair encoded by the nucleic acid sequence pair of SEQ ID NO:71/79.

In a further embodiment, the antibody or fragment thereof further comprises a HCDR1 domain encoded by a nucleotide sequence selected from the group consisting of SEQ ID NO: 3, 19, 35, 51, 67, 83, 99, 115, 131, 147 and 181, or a substantially identical sequence having at least 90%, at least 95%, at least 98%, or at least 99% homology thereof; and a HCDR2 domain encoded by a nucleotide sequence selected from the group consisting of SEQ ID NO:5, 21, 37, 53, 69, 85, 101, 117, 133, 149 and 183, or a substantially identical sequence having at least 90%, at least 95%, at least 98%, or at least 99% homology thereof; and optionally further comprises a LCDR1 domain encoded by a nucleotide sequence selected from the group consisting of SEQ ID NO:11, 27, 43, 59, 75, 91, 107, 123, 139, 155 and 189, or a substantially identical sequence having at least 90%, at least 95%, at least 98%, or at least 99% homology thereof; and/or a LCDR2 domain encoded by a nucleotide sequence selected from the group consisting of sequences: gctgcatcc, aaggcgtct, gctgcatcc, aaggcgtct, aaggcgtct, actacttcc, cctgcatcc, actgcatcc, aaggtttct, gttgcagccand aaggcgtct, or a substantially identical sequence having at least 90%, at least 95%, at least 98%, or at least 99% homology thereof.

Alternatively, the invention features an antibody or antigen-binding fragment of an antibody comprising a HCDR1/HCDR2/HCDR3 combination encoded by a nucleotide sequence combination selected from the group consisting of SEQ ID NO:3/5/7, 19/21/23, 35/37/39, 51/53/55, 67/69/71, 83/85/87, 99/101/103, 115/117/119, 131/133/135, 147/149/151 and 181/183/185; and/or a LCDR1/LCDR2/LCDR3 combination encoded by a nucleotide sequence combination selected from the group consisting of SEQ ID NO:11/gctgcatcc/15, 27/aaggcgtct/31, 43/gctgcatcc/47, 59/aaggcgtct/63, 75/aaggcgtct/79, 91/actacttcc/95, 107/cctgcatcc/111, 123/actgcatcc/127, 139/aaggtttct/143, 155/gttgcagcc/159 and 189/aaggcgtct/193. In one embodiment, the antibody or fragment thereof comprises heavy and light chain CDR sequences encoded by a nucleotide sequence combination of SEQ ID NO:67/69/71/75/aaggcgtct/79.

In a fifth aspect, the invention features a human anti-ANGPTL3 antibody or antigen-binding fragment thereof comprising a heavy chain variable region (HCVR) encoded by nucleotide sequence segments derived from V_{H}, D_{H} and J_{H} germline sequences, and a light chain variable region (LCVR) encoded by nucleotide sequence segments derived from V_{K} and J_{K} germline sequences, wherein the HCVR and the LCVR are encoded by nucleotide sequence segments derived from a germline gene combination selected from the group consisting of: (i) V_{H}3-43, D_{H}3-3, J_{H}3, V_{K}1-5 and J_{K}2; (ii) V_{H}3-11, D_{H}1-1, J_{H}4, V_{K}1-39 and J_{K}4; (iii) V_{H}3-30, D_{H}1-7, J_{H}6, V_{K}1-5 and J_{K}1; (iv) V_{H}3-30, D_{H}1-26, J_{H}6, V_{K}1-12 and J_{K}3; (v) V_{H}3-30, D_{H}3-10, J_{H}6, V_{K}1-12 and J_{K}3; and (vi) V_{H}3-23, D_{H}3-10, J_{H}4, V_{K}1-5 and J_{K}1.

In a sixth aspect, the invention features an antibody or antigen-binding fragment thereof that specifically binds to hANGPTL3 with an equilibrium dissociation constant (K_{D}) of about 7 nM or less, about 6 nM or less, about 5 nM or less, about 4 nM or less, about 3 nM or less, about 2 nM or less, or about 1 nM or less, as measured by surface plasmon resonance assay (for example, BIACORE™). In certain embodiments, the antibody of the invention exhibits a K_{D} of about 800 pM or less, about 700 pM or less; about 600 pM or less; about 500 pM or less; about 400 pM or less; about 300 pM or less; about 200 pM or less; about 100 pM or less; or about 50 pM or less.

In a seventh aspect, the present invention provides an anti-hANGPTL3 antibody or antigen-binding fragment thereof that binds hANGPTL3 protein of SEQ ID NO:161, but does not cross-react with a related protein, such as a human angiopoietin-like protein 4 (hANGPTL4; SEQ ID NO:164), as determined by, for example, ELISA, surface plasmon resonance assay, or LUMINEX® XMAP® Technology, as described herein. ANGPTL4 is another secreted protein that is known to reduce LPL activity and has an N-terminal coiled-coil region and a C-terminal fibrinogen-like domain (Ge, et al., 2004, J Biol Chem 279:2038-2045; Yau, et al., 2009, J Biol Chem 284:11942-11952). In related embodiments, the invention provides an anti-hANGPTL3 antibody or antigen binding fragment thereof that binds a hANGPTL3 protein and cross-reacts with a hANGPTL4 protein. In certain embodiments, the binding affinity of the hANGPTL3 antibody or fragment thereof to hANGPTL4 protein is about 75% or less, or about 50% or less, of the binding affinity of the antibody or fragment to the hANGPTL3 protein.

In another related embodiment, the invention provides an anti-hANGPTL3 antibody or antigen binding fragment thereof that does not cross-react with mouse ANGPTL3 (mANGPTL3; SEQ ID NO:163), or rat ANGPTL3 (rANGPTL3; SEQ ID NO:175), but does cross-react with cynomolgus monkey (Macaca fascicularis) ANGPTL3 (MfANGPTL3), for example, with the N-terminal 17-170 residues of SEQ ID NO:177 (a partial amino acid sequence of MfANGPTL3). In yet another related embodiment, the invention provides an anti-hANGPTL3 antibody or fragment thereof that cross-reacts with MfANGPTL3, mANGPTL3 and rANGTPL3.

The invention encompasses anti-hANGPTL3 antibodies having a modified glycosylation pattern. In some applications, modification to remove undesirable glycosylation sites may be useful, or e.g., removal of a fucose moiety to increase antibody dependent cellular cytotoxicity (ADCC) function (see Shield et al. (2002) JBC 277:26733). In other applications, removal of N-glycosylation site may reduce undesirable immune reactions against the therapeutic antibodies, or increase affinities of the antibodies. In yet other applications, modification of galactosylation can be made in order to modify complement dependent cytotoxicity (CDC).

In an eighth aspect, the invention features a pharmaceutical composition comprising a recombinant human antibody or fragment thereof which specifically binds hANGPTL3 and a pharmaceutically acceptable carrier. In one embodiment, the invention provides a pharmaceutical composition comprising one or more anti-ANGPTL3 antibodies or fragments thereof of the invention, which do not cross-compete with one another, and a pharmaceutically acceptable carrier. In one embodiment, the pharmaceutical composition of the invention can contain two or more non-blocking antibodies, which do not compete with one another for specific binding to hANGPTL3 and are effective in clearing hANGPTL3 from the circulation. Suitable combinations of non-blocking antibodies include, but are not limited to, a combination of antibodies comprising HCVR and LCVR sequence pairs (HCVR/LCVR) of: (i) SEQ ID NO:82/90 and 180/188, respectively; (ii) SEQ ID NO:114/122 and 180/188, respectively; (iii) SEQ ID NO:82/90 and 18/26, respectively; or (iv) SEQ ID NO:114/122 and 18/26, respectively.

In related embodiments, the invention features a composition which is a combination of an antibody or antigen-binding fragment thereof of the invention, and a second therapeutic agent. The second therapeutic agent may be one or more of any agent such as (1) 3-hydroxy-3-methylglutaryl-coenzyme A (HMG-CoA) reductase inhibitors, such as cerivastatin, atorvastatin, simvastatin, pitavastatin, rosuvastatin, fluvastatin, lovastatin, pravastatin, and the like; (2) inhibitors of cholesterol uptake and/or bile acid re-absorption; (3) niacin, which increases lipoprotein catabolism (4) fibrates or amphipathic carboxylic acids, which reduce low-density lipoprotein (LDL) level, improve high-density lipoprotein (HDL) and TG levels, and reduce the number of non-fatal heart attacks; and (5) activators of the LXR transcription factor that plays a role in cholesterol elimination such as 22-hydroxycholesterol, or fixed combinations such as ezetimibe plus simvastatin; a statin with a bile resin (e.g., cholestyramine, colestipol, colesevelam), a fixed combination of niacin plus a statin (e.g., niacin with lovastatin); or with other lipid lowering agents such as omega-3-fatty acid ethyl esters (for example, omacor). Furthermore, the second therapeutic agent can be one or more other inhibitors of ANGPTL3 as well as inhibitors of other molecules, such as ANGPTL4, ANGPTL5, ANGPTL6 and proprotein convertase subtilisin/kexin type 9 (PCSK9), which are involved in lipid metabolism, in particular, cholesterol and/or triglyceride homeostasis. Inhibitors of these molecules include small molecules and antibodies that specifically bind to these molecules and block their activity.

In related embodiments, the second therapeutic agent may be one or more anti-cancer agents, such as chemotherapeutic agents, anti-angiogenic agents, growth inhibitory agents, cytotoxic agents, apoptotic agents, and other agents well known in the art to treat cancer or other proliferative diseases or disorders, as well as other therapeutic agents, such as analgesics, anti-inflammatory agents, including non-steroidal anti-inflammatory drugs (NSAIDS), such as Cox-2 inhibitors, and the like, so as to ameliorate and/or reduce the symptoms accompanying the underlying cancer/tumor.

In a ninth aspect, the invention features methods for neutralizing, inhibiting, blocking, abrogating, reducing or interfering with, hANGPTL3 activity using one or more anti-hANGPTL3 antibodies or antigen-binding fragments thereof of the invention. In one embodiment, the invention provides a therapeutic method comprising administering to a subject in need thereof a therapeutically effective amount of a pharmaceutical composition comprising one or more anti-hANGPTL3 antibodies or antigen-binding fragments thereof of the invention and, optionally one or more additional therapeutic agents described above. The anti-ANGPTL3 antibodies or fragments thereof of the invention may be neutralizing antibodies or non-blocking antibodies against ANGPTL3, or combinations thereof.

In related embodiments, the invention provides methods of enhancing the clearance of hANGPTL3 from the circulation of a subject in need thereof, comprising administering to the subject at least two anti-hANGPTL3 antibodies or fragments thereof of the invention that do not compete with one another for binding to hANGPTL3 and preferably do not block at least one activity of hANGPTL3 (i.e., non-blocking antibodies). At least one activity of hANGPTL3 referred to includes, but not the way of limitation, inhibiting LPL activity, inducing angiogenesis, and the like. In one embodiment, a combination of at least two non-blocking anti-hANGPTL3 antibodies or fragments thereof enhances the clearance of hANGPTL3 from the circulation by at least about 20%, about 30%, about 40%, about 50%, about 60%, about 70%, or about 80%, relative to not administering the antibodies or fragments. Circulating levels of hANGPTL3 can be measured by in vitro assays well known in the art and those described herein. In another embodiment, the combination of at least two non-blocking anti-hANGPTL3 antibodies comprises HCVR and LCVR sequence pairs (HCVR/LCVR) of: (i) SEQ ID NO:82/90 and 180/188, respectively; (ii) SEQ ID NO:114/122 and 180/188, respectively; (iii) SEQ ID NO:82/90 and 18/26, respectively; or (iv) SEQ ID NO:114/122 and 18/26, respectively.

The disease or disorder treatable by the methods of the invention is any disease or condition which is improved, ameliorated, inhibited or prevented, or its occurrence rate reduced, compared to that without anti-hANGPTL3 antibody treatment (e.g., ANGPTL3-mediated diseases or disorders), by removing, inhibiting, reducing, or otherwise interfering with, ANGPTL3 activity. Examples of diseases or disorders treatable by the methods of the invention include, but are not limited to, those involving lipid metabolism, such as hyperlipidemia, hyperlipoproteinemia and dyslipidemia, including atherogenic dyslipidemia, diabetic dyslipidemia, hypertriglyceridemia, including severe hypertriglyceridemia with TG>1000 mg/dL, hypercholesterolemia, chylomicronemia, mixed dyslipidemia (obesity, metabolic syndrome, diabetes, etc.), lipodystrophy, lipoatrophy, and the like, which are caused by, for example, decreased LPL activity and/or LPL deficiency, decreased LDL receptor (LDLR) activity and/or LDL receptor deficiency (e.g., homozygous familial hypercholesterolemia with LDLR^{-/-}), altered ApoC2, ApoE deficiency, increased ApoB, increased production and/or decreased elimination of very low-density lipoprotein (VLDL), certain drug treatment (e.g., glucocorticoid treatment-induced dyslipidemia), any genetic predisposition, diet, life style, and the like. The methods of the invention can also prevent or treat diseases or disorders associated with or resulting from hyperlipidemia, hyper-lipoproteinemia, and/or dyslipidemia, including, but not limited to, cardiovascular diseases or disorders, such as atherosclerosis, aneurysm, hypertension, angina, stroke, cerebrovascular diseases, congestive heart failure, coronary artery diseases, myocardial infarction, peripheral vascular diseases, and the like; acute pancreatitis; nonalcoholic steatohepatitis (NASH); blood sugar disorders, such as diabetes; obesity, and the like.

Other examples of diseases or disorders treatable by the methods of the invention include cancer/tumor as well as non-neoplastic angiogenesis-associated diseases or disorders, including ocular angiogenic diseases or disorders, such as age-related macular degeneration, central retinal vein occlusion or branch retinal vein occlusion, diabetic retinopathy, retinopathy of prematurity, and the like, inflammatory diseases or disorders, such as arthritis, rheumatoid arthritis (RA), psoriasis, and the like.

Other embodiments will become apparent from a review of the ensuing detailed description.

### BRIEF DESCRIPTION OF THE FIGURES

FIG. 1 shows a sequence alignment of Peptides 1-3 used in an anti-hANGPTL3 antibody binding experiment (Example 5) against the relevant portions of hANGPTL3 sequence (i.e., within residues 30 to 70 of SEQ ID NO:161 or GenBank #NP_055310). Peptide 1 (control: ANGPTL4 peptide; SEQ ID NO:168); Peptide 2 (ANGPTL3 peptide; SEQ ID NO:169); and Peptide 3 (ANGPTL3 peptide; SEQ ID NO:170).

FIG. 2 shows the results of anti-hANGPTL3 antibody binding to the N-terminal coiled-coil peptides of hANGPTL3 (Peptides 2 and 3) or hANGPTL4 (Peptide 1). [symbol] : Isotype Control; and [symbol] : H4H1276S antibody.

### DETAILED DESCRIPTION

Before the present invention is described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, preferred methods and materials are now described. All publications mentioned herein are incorporated herein by reference in their entirety.

### Definitions

The term “human angiopoietin-like protein 3” or “hANGPTL3”, as used herein, refers to ANGPTL3 having the nucleic acid sequence shown in SEQ ID NO: 162 and the amino acid sequence of SEQ ID NO: 161, or a biologically active fragment thereof.

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (HCVR) and a heavy chain constant region (C_{H}; comprised of domains C_{H}1, C_{H}2 and C_{H}3). Each light chain is comprised of a light chain variable region (LCVR) and a light chain constant region (C_{L}). The HCVR and LCVR can be further subdivided into regions of hypervariability, termed complementarity determining regions (CDR), interspersed with regions that are more conserved, termed framework regions (FR). Each HCVR and LCVR is composed of three CDRs and four FRs, arranged from amino-terminus to carboxy-terminus in the following order: FR1, CDR1, FR2, CDR2, FR3, CDR3, and FR4.

Substitution of one or more CDR residues or omission of one or more CDRs is also possible. Antibodies have been described in the scientific literature in which one or two CDRs can be dispensed with for binding. Padlan, et al., (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos, et al., 2002 J Mol Biol 320:415-428).

CDR residues not contacting antigen can be identified based on previous studies (for example, residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The term “human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences.

The fully-human anti-hANGPTL3 antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residues(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline back-mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residues of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The present invention also includes anti-ANGPTL3 antibodies comprising variants of any of the HCVR, LCVR, and/or CDR amino acid sequences disclosed herein having one or more conservative substitutions. For example, the present invention includes anti-ANGPTL3 antibodies having HCVR, LCVR, and/or CDR amino acid sequences with, e.g., 10 or fewer, 8 or fewer, 6 or fewer, 4 or fewer, 2 or 1, conservative amino acid substitution(s) relative to any of the HCVR, LCVR, and/or CDR amino acid sequences disclosed herein. In one embodiment, a HCVR comprises the amino acid sequence of SEQ ID NO:66 with 10 or fewer conservative amino acid substitutions therein. In another embodiment, a HCVR comprises the amino acid sequence of SEQ ID NO: 66 with 8 or fewer conservative amino acid substitutions therein. In another embodiment, a HCVR comprises the amino acid sequence of SEQ ID NO:66 with 6 or fewer conservative amino acid substitutions therein. In another embodiment, a HCVR comprises the amino acid sequence of SEQ ID NO:66 with 4 or fewer conservative amino acid substitutions therein. In yet another embodiment, a HCVR comprises the amino acid sequence of SEQ ID NO:66 with 2 or 1 conservative amino acid substitution(s) therein. In one embodiment, a LCVR comprises the amino acid sequence of SEQ ID NO:74 with 10 or fewer conservative amino acid substitutions therein. In another embodiment, a LCVR comprises the amino acid sequence of SEQ ID NO:74 with 8 or fewer conservative amino acid substitutions therein. In another embodiment, a LCVR comprises the amino acid sequence of SEQ ID NO:74 with 6 or fewer conservative amino acid substitutions therein. In another embodiment, a LCVR comprises the amino acid sequence of SEQ ID NO:74 with 4 or fewer conservative amino acid substitutions therein. In yet another embodiment, a LCVR comprises the amino acid sequence of SEQ ID NO:74 with 2 or 1 conservative amino acid substitution(s) therein.

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-display antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

In certain embodiments, antibody or antibody fragments of the invention may be conjugated to a therapeutic moiety (“immunoconjugate”), such as a cytotoxin, a chemotherapeutic drug, an immunosuppressant or a radioisotope.

The term “specifically binds,” or the like, means that an antibody or antigen-binding fragment thereof forms a complex with an antigen that is relatively stable under physiological conditions. Specific binding can be characterized by an equilibrium dissociation constant (K_{D}) of about 1×10⁻⁶ M or less (i.e., a smaller K_{D} denotes a tighter binding). Methods for determining whether two molecules specifically bind are well known in the art and include, for example, equilibrium dialysis, surface plasmon resonance, and the like. An isolated antibody that specifically binds hANGPTL3 may, however, exhibit cross-reactivity to other antigens, such as ANGPTL3 molecules from other species, for example, cynomolgus monkey ANGPTL3, mouse ANGPTL3, rat ANGPTL3, and/or hANGPTL4 having the amino acid sequence of SEQ ID NO: 164. Moreover, multi-specific antibodies (e.g., bispecifics) that bind to hANGPTL3 and one or more additional antigens are nonetheless considered antibodies that “specifically bind” hANGPTL3, as used herein.

The term “high affinity” antibody refers to those antibodies having a binding affinity to hANGPTL3, expressed as K_{D}, of about 2×10⁻⁹ M or less, about 1.5×10⁻⁹ M or less, about 1×10⁻⁹ M or less, about 0.5×10⁻⁹ M or less, about 0.25×10⁻⁹ M or less, about 1×10⁻¹⁰ M or less, or about 0.5×10⁻¹⁰ M or less, as measured by surface plasmon resonance, e.g., BIACORE™ or solution-affinity ELISA.

The term “K_{D}”, as used herein, is intended to refer to the equilibrium dissociation constant of a particular antibody-antigen interaction.

By the term “slow off rate”, “Koff” or “k_{d}” is meant an antibody that dissociates from hANGPTL3 with a rate constant of 4×10⁻³ s⁻¹ or less, 3×10⁻³ s⁻¹ or less, 2×10⁻³ s⁻¹ or less, 1×10⁻³ s⁻¹ or less, 1×10⁻⁴ s^{31 1} or less, as determined by surface plasmon resonance, e.g., BIACORE™.

By the term “intrinsic affinity constant” or “kₐ” is meant an antibody that associates with hANGPTL3 at a rate constant of about 1×10³ M⁻¹s⁻¹ or higher, as determined by surface plasmon resonance, e.g., BIACORE™.

An “isolated antibody”, as used herein, is intended to refer to an antibody that is substantially free of other mAbs having different antigenic specificities (e.g., an isolated antibody that specifically binds hANGPTL3 is substantially free of mAbs that specifically bind antigens other than hANGPTL3). An isolated antibody that specifically binds hANGPTL3 may, however, have cross-reactivity to other antigens, such as ANGPTL3 molecules from other species, such as cynomolgus monkey, mouse, rat, and/or other related proteins, such as human ANGPTL4.

A “neutralizing”, “blocking” or “abrogating” antibody, as used herein (or an antibody that “neutralizes”, “blocks” or “abrogates” ANGPTL3 activity), is intended to refer to an antibody whose binding to ANGPTL3 results in direct inhibition of at least one biological activity of ANGPTL3, as assessed by standard in vitro assays known in the art (for example, see Examples below). The terms, “neutralize”, “inhibit”, “block” and “abrogate”, may be used herein interchangeably. A “non-blocking” antibody refers to an antibody whose binding to ANGPTL3 does not directly block a targeted activity of ANGPTL3 as assessed by standard in vitro assays, but yet may be an “interfering” antibody whose binding to ANGPTL3 results in indirect inhibition, reduction, attenuation, or other interference, of at least one biological activity of ANGPTL3 in vivo, e.g., by enhancing the clearance of ANGPTL3 from the circulation. Clearance of ANGPTL3 from the circulation can be particularly enhanced by a combination of at least two non-blocking antibodies. The neutralization, inhibition, abrogation, reduction, attenuation or interference, of a biological activity of ANGPTL3 can be assessed by measuring one or more indicators of ANGPTL3 biological activity by one or more of several standard in vitro or in vivo assays known in the art (also see Examples below).

The term “surface plasmon resonance”, as used herein, refers to an optical phenomenon that allows for the analysis of real-time biospecific interactions by detection of alterations in protein concentrations within a biosensor matrix, for example using the BIACORE™ system (Pharmacia Biosensor AB, Uppsala, Sweden and Piscataway, N.J.).

The term “epitope” is a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet, et al., (1992) Science 256: 1443 45. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul, et al., (1990) J. Mol. Biol. 215:403 410 and (1997) Nucleic Acids Res. 25:3389 402.

By the phrase “therapeutically effective amount” is meant an amount that produces the desired effect for which it is administered. The exact amount will depend on the purpose of the treatment, the age and the size of a subject treated, the route of administration, and the like, and will be ascertainable by one skilled in the art using known techniques (see, for example, Lloyd (1999) The Art, Science and Technology of Pharmaceutical Compounding).

### Preparation of Human Antibodies

Methods for generating human antibodies in transgenic mice are known in the art. Any such known methods can be used in the context of the present invention to make human antibodies that specifically bind to ANGPTL3.

Using VELOCIMMUNE™ technology or any other known method for generating monoclonal antibodies, high affinity chimeric antibodies to ANGPTL3 are initially isolated having a human variable region and a mouse constant region. As in the experimental section below, the antibodies are characterized and selected for desirable characteristics, including affinity, selectivity, epitope, and the like.

In general, the antibodies of the instant invention possess high affinities, typically possessing K_{D} of from about 10⁻¹² M through about 10⁻⁹ M, when measured by binding to antigen either immobilized on solid phase or in solution phase. The mouse constant regions are replaced with desired human constant regions, for example, wild-type IgG1 or IgG4, or modified IgG1 or IgG4, to generate the fully human antibodies of the invention. While the constant region selected may vary according to specific use, high affinity antigen-binding and target specificity characteristics of the antibodies reside in the variable region.

### Epitope Mapping and Related Technologies

To screen for antibodies that bind to a particular epitope, a routine cross-blocking assay such as that described in Antibodies, Harlow and Lane (Cold Spring Harbor Press, Cold Spring Harb., NY) can be performed. Other methods include alanine scanning mutants, peptide blots (Reineke (2004) Methods Mol Biol 248:443-63), or peptide cleavage analysis. In addition, methods such as epitope excision, epitope extraction and chemical modification of antigens can be employed (Tomer (2000) Protein Science 9: 487-496).

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (US 2004/0101920). Each category may reflect a unique epitope either distinctly different from or partially overlapping with epitope represented by another category. This technology allows rapid filtering of genetically identical mAbs, such that characterization can be focused on genetically distinct mAbs. When applied to hybridoma screening, MAP may facilitate identification of rare hybridoma clones that produce mAbs having the desired characteristics. MAP may be used to sort the anti-ANGPTL3 mAbs of the invention into groups of mAbs binding different epitopes.

ANGPTL3 contains an amino-terminal coiled-coil domain and a carboxyl-terminal fibrinogen like domain (FD) and the ANGPTL3 protein forms an oligomer in the absence of intermolecular disulfide bonds (Ge, et al., 2005, J Lipid Res 46:1484-1490). It has been reported that the N-terminal coiled-coil domain is important in the inhibition of LPL activity (Ono, et al., 2003, J Biol Chem 278:41804-41809). Thus, in certain embodiments, the anti-hANGPTL3 antibody or antigen-binding fragment of an antibody binds an epitope within the N-terminal coiled-coil domain (residues 17-209) of hANGPTL3 (SEQ ID NO:161) and neutralizes at least one activity of hANGPTL3 (e.g., inhibition of LPL activity). In another embodiments, the anti-hANGPTL3 antibody or antigen-binding fragment thereof binds an epitope within the N-terminal coiled-coil domain of hANGPTL3 and neutralizes at least one activity of hANGPTL3, with the proviso that the antibody or fragment thereof does not bind to the ANGPTL3 peptide of SEQ ID NO:170. In one embodiment, the antibody or fragment thereof specifically binds an epitope within residues 17 to 200, 17 to 100, 17 to 70, 17 to 65, 17 to 60, 17 to 57, 17 to 55, 17 to 50, 17 to 45, 17 to 40, or 17 to 35, of hANGPTL3 (SEQ ID NO:161), optionally with the proviso that the antibody or fragment thereof does not bind to the ANGPTL3 peptide of SEQ ID NO:170. In another embodiment, the antibody or fragment thereof specifically binds an epitope within residues 40 to 200, 40 to 100, 40 to 70, 50 to 200, 50 to 100, 50to 70, 58 to 200, 58 to 100, 58 to 70, 58 to 68, or 61 to 66 (known as a “heparin-binding motif”) of hANGPTL3 (SEQ ID NO:161), optionally with the proviso that the antibody or fragment thereof does not bind to the ANGPTL3 peptide of SEQ ID NO:170. In some embodiments, the antibody or antibody fragment binds an epitope which may involve more than one of the enumerated epitopes or residues within the N-terminal coiled-coil region of hANGPTL3, optionally with the proviso that the antibody or fragment thereof does not bind to the ANGPTL3 peptide of SEQ ID NO:170.

In other embodiments, hANGPTL3 antibody or fragment thereof binds one or more fragments of hANGPTL3, for example, a fragment of at least 5 residues, at least 7 residues, at least 10 residues, at least 20 residues, at least 30 residues, at least 50 residues, at least 70 residues, at least 100 residues, at least 150 residues, or at least 200 residues, of hANGPTL3 (SEQ ID NO:161), optionally with the proviso that the antibody or fragment thereof does not bind to the ANGPTL3 peptide of SEQ ID NO:170.

The present invention includes hANGPTL3 antibodies that bind to the same epitope as any of the specific exemplary antibodies described herein. Likewise, the present invention also includes anti-hANGPTL3 antibodies that compete for binding to hANGPTL3 or a hANGPTL3 fragment with any of the specific exemplary antibodies described herein.

One can easily determine whether an antibody binds to the same epitope as, or competes for binding with, a reference anti-hANGPTL3 antibody by using routine methods known in the art. For example, to determine if a test antibody binds to the same epitope as a reference anti-hANGPTL3 antibody of the invention, the reference antibody is allowed to bind to a hANGPTL3 protein or peptide under saturating conditions. Next, the ability of a test antibody to bind to the hANGPTL3 molecule is assessed. If the test antibody is able to bind to hANGPTL3 following saturation binding with the reference anti-hANGPTL3 antibody, it can be concluded that the test antibody binds to a different epitope than the reference anti-hANGPTL3 antibody. On the other hand, if the test antibody is not able to bind to the hANGPTL3 molecule following saturation binding with the reference anti-hANGPTL3 antibody, then the test antibody may bind to the same epitope as the epitope bound by the reference anti-hANGPTL3 antibody of the invention.

To determine if an antibody competes for binding with a reference anti-hANGPTL3 antibody, the above-described binding methodology is performed in two orientations: In a first orientation, the reference antibody is allowed to bind to a hANGPTL3 molecule under saturating conditions followed by assessment of binding of the test antibody to the hANGPTL3 molecule. In a second orientation, the test antibody is allowed to bind to a hANGPTL3 molecule under saturating conditions followed by assessment of binding of the reference antibody to the ANGPTL3 molecule. If, in both orientations, only the first (saturating) antibody is capable of binding to the ANGPTL3 molecule, then it is concluded that the test antibody and the reference antibody compete for binding to hANGPTL3. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20-or 100-fold excess of one antibody inhibits binding of the other by at least 50% but preferably 75%, 90% or even 99% as measured in a competitive binding assay (see, e.g., Junghans, et al., Cancer Res, 1990:50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

### Immunoconjugates

The invention encompasses a human anti-ANGPTL3 monoclonal antibody conjugated to a therapeutic moiety (“immunoconjugate”), such as a cytotoxin, a chemotherapeutic drug, an immunosuppressant or a radioisotope. Cytotoxin agents include any agent that is detrimental to cells. Examples of suitable cytotoxin agents and chemotherapeutic agents for forming immunoconjugates are known in the art, see for example, WO 05/103081.

### Bispecifics

The antibodies of the present invention may be monospecific, bispecific, or multispecific. Multispecific mAbs may be specific for different epitopes of one target polypeptide or may contain antigen-binding domains specific for more than one target polypeptide. See, e.g., Tutt, et al,. (1991) J. Immunol. 147:60-69. The human anti-hANGPTL3 mAbs can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment, to produce a bispecific or a multispecific antibody with a second binding specificity.

An exemplary bi-specific antibody format that can be used in the context of the present invention involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bi-specific antibody format described above are contemplated within the scope of the present invention.

### Bioequivalents

The anti-hANGPTL3 antibodies and antibody fragments of the present invention encompass proteins having amino acid sequences that vary from those of the described mAbs, but that retain the ability to bind human ANGPTL3. Such variant mAbs and antibody fragments comprise one or more additions, deletions, or substitutions of amino acids when compared to parent sequence, but exhibit biological activity that is essentially equivalent to that of the described mAbs. Likewise, the anti-hANGPTL3 antibody-encoding DNA sequences of the present invention encompass sequences that comprise one or more additions, deletions, or substitutions of nucleotides when compared to the disclosed sequence, but that encode an anti-hANGPTL3 antibody or antibody fragment that is essentially bioequivalent to an anti-hANGPTL3 antibody or antibody fragment of the invention. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied. In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-hANGPTL3 antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation.

### Therapeutic Administration and Formulations

The invention provides therapeutic compositions comprising the anti-hANGPTL3 antibodies or antigen-binding fragments thereof of the present invention and the therapeutic methods using the same. The administration of therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell, et al., “Compendium of excipients for parenteral formulations” PDA (1998) J Pharm Sci Technol 52:238-311.

The dose may vary depending upon the age and the size of a subject to be administered, target disease, the purpose of the treatment, conditions, route of administration, and the like. When the antibody of the present invention is used for treating various conditions and diseases directly or indirectly associated with ANGPTL3, including hypercholesterolemia, disorders associated with LDL and apolipoprotein B, and lipid metabolism disorders, and the like, in an adult patient, it is advantageous to intravenously or subcutaneously administer the antibody of the present invention at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. In certain embodiments, the antibody or antigen-binding fragment thereof of the invention can be administered as an initial dose of at least about 0.1 mg to about 800 mg, about 1 to about 500 mg, about 5 to about 300 mg, or about 10 to about 200 mg, to about 100 mg, or to about 50 mg. In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu, et al. (1987) J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition can be also delivered in a vesicle, in particular a liposome (see Langer (1990) Science 249:1527-1533; Treat, et al., (1989) in Liposomes in the Therapy of Infectious Disease and Cancer, Lopez Berestein and Fidler (eds.), Liss, New York, pp. 353-365; Lopez-Berestein, ibid., pp. 317-327; see generally ibid.).

In certain situations, the pharmaceutical composition can be delivered in a controlled release system. In one embodiment, a pump may be used (see Langer, supra; Sefton (1987) CRC Crit. Ref. Biomed. Eng. 14:201). In another embodiment, polymeric materials can be used; see, Medical Applications of Controlled Release, Langer and Wise (eds.), CRC Pres., Boca Raton, Florida (1974). In yet another embodiment, a controlled release system can be placed in proximity of the composition's target, thus requiring only a fraction of the systemic dose (see, e.g., Goodson, in Medical Applications of Controlled Release, supra, vol. 2, pp. 115-138, 1984).

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule. A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but certainly are not limited to the SOLOSTAR™ pen (Sanofi-Aventis), the FLEXPEN™ (Novo Nordisk), and the KWIKPEN™ (Eli Lilly).

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 0.1 to about 800 mg per dosage form in a unit dose; especially in the form of injection, the aforesaid antibody is contained in about 1 to about 500 mg, in about 5 to 300 mg, in about 8 to 200 mg, and in about 10 to about 100 mg for the other dosage forms.

### Combination Therapies

The invention further provides therapeutic methods for treating diseases or disorders, which is directly or indirectly associated with hANGPTL3, by administering a hANGPTL3 antibody or fragment thereof of the invention in combination with one or more additional therapeutic agents. The additional therapeutic agent may be one or more of any agent that is advantageously combined with one or more antibodies or fragments thereof of the invention, including HMG-CoA reductase inhibitors, such as cerovastatin, atorvastatin, simvastatin, pitavastin, rosuvastatin, fluvastatin, lovastatin, pravastatin, and the like; niacin; various fibrates, such as fenofibrate, bezafibrate, ciprofibrate, clofibrate, gemfibrozil, and the like; LXR transcription factor activators, and the like. Furthermore, the hANGPTL3 antibody or fragment thereof of the invention can be co-administered with other ANGPTL3 inhibitors as well as inhibitors of other molecules, such as ANGPTL4,ANGPTL5, ANGPTL6 and proprotein convertase subtilisin/kexin type 9 (PCSK9), which are involved in lipid metabolism, in particular, cholesterol and/or triglyceride homeostasis. Inhibitors of these molecules include small molecules and antibodies that specifically bind to these molecules and block their activity (see, for example, anti-PCSK9 antibodies disclosed in U.S. 2010/0166768 A1).

Furthermore, the additional therapeutic agent may be one or more anti-cancer agents, such as chemotherapeutic agents, anti-angiogenic agents, growth inhibitory agents, cytotoxic agents, apoptotic agents, and other agents well known in the art to treat cancer or other proliferative diseases or disorders. Examples of anti-cancer agents include, but are not limited to, an anti-mitotic agent, such as docetaxel, paclitaxel, and the like; a platinum-based chemotherapeutic compound, such as cisplatin, carboplatin, iproplatin, oxaliplatin, and the like; or other conventional cytotoxic agent, such as 5-fluorouracil, capecitabine, irinotecan, leucovorin, gemcitabine, and the like, and anti-angiogenic agents, including vascular endothelial growth factor (VEGF) antagonists, such as anti-VEGF antibodies, e.g., bevacizumab (AVASTIN®, Genentech) and a receptor-based blocker of VEGF, e.g., “VEGF trap” described in U.S. Pat. No. 7,070,959, delta-like ligand 4 (DII4) antagonists, such as anti-DII4 antibodies as described in U.S. Patent Application Publication No. 2008/0181899, and a fusion protein containing the extracellular domain of DII4, e.g., DII4-Fc as described in U.S. Patent Application Publication No. 2008/0107648; inhibitors of receptor tyrosine kinases and/or angiogenesis, including sorafenib (NEXAVAR® by Bayer Pharmaceuticals Corp.), sunitinib (SUTENT® by Pfizer), pazopanib (VOTRIENT™ by GlaxoSmithKline), toceranib (PALLADIA™ by Pfizer), vandetanib (ZACTIMA™ by AstraZeneca), cediranib (RECENTIN® by AstraZeneca), regorafenib (BAY 73-4506 by Bayer), axitinib (AG013736 by Pfizer), lestaurtinib (CEP-701 by Cephalon), erlotinib (TARCEVA® by Genentech), gefitinib (IRESSA™ by AstraZeneca), BIBW 2992 (TOVOK™ by Boehringer Ingelheim), lapatinib (TYKERB® by GlaxoSmithKline), neratinib (HKI-272 by Wyeth/Pfizer), and the like, and pharmaceutically acceptable salts, acids or derivatives of any of the above. In addition, other therapeutic agents, such as analgesics, anti-inflammatory agents, including non-steroidal anti-inflammatory drugs (NSAIDS), such as Cox-2 inhibitors, and the like, may be also co-administered with the hANGPTL3 antibody or fragment thereof of the invention so as to ameliorate and/or reduce the symptoms accompanying the underlying cancer/tumor.

The hANGPTL3 antibody or fragment thereof of the invention and the additional therapeutic agent(s) can be co-administered together or separately. Where separate dosage formulations are used, the antibody or fragment thereof of the invention and the additional agents can be administered concurrently, or separately at staggered times, i.e., sequentially, in appropriate orders.

### Diagnostic Uses of the Antibodies

The anti-ANGPTL3 antibodies of the present invention can be also used to detect and/or measure ANGPTL3 in a sample, e.g., for diagnostic purposes. For example, an anti-ANGPTL3 Ab or fragment thereof, can be used to diagnose a condition or disease characterized by aberrant expression (e.g., over-expression, under-expression, lack of expression, etc.) of ANGPTL3.Exemplary diagnostic assays for ANGPTL3 may comprise, e.g., contacting a sample obtained from a patient, with an anti-ANGPTL3 Ab of the invention, wherein the anti-ANGPTL3 antibody is labeled with a detectable label or reporter molecule or used to selectively capture and isolate ANGPTL3protein from patient samples. Alternatively, an unlabeled anti-ANGPTL3 Ab can be used in diagnostic applications in combination with a secondary antibody which is itself detectably labeled. The detectable label or reporter molecule can be a radioisotope, such as ³H, ¹⁴C, ³²P, ³⁵S, ¹³¹I or ¹²⁵I; a fluorescent or chemiluminescent moiety, such as fluorescein isothiocyanate, or rhodamine; or an enzyme such as alkaline phosphatase, β-galactosidase, horseradish peroxidase, or luciferase. Assays that can be used to detect or measure ANGPTL3 in a sample include enzyme-linked immunosorbent assay (ELISA), radioimmunoassay (RIA), fluorescence-activated cell sorting (FACS), and the like.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used but some experimental errors and deviations should be accounted for. Unless indicated otherwise, molecular weight is average molecular weight, temperature is in degrees Centigrade, and pressure is at or near atmospheric.

### Example 1: Generation of Human Antibodies to Human ANGPTL3

VELOCIMMUNE™ mice were immunized with human ANGPTL3, and the antibody immune response monitored by antigen-specific immunoassay using serum obtained from these mice. Anti-hANGPTL3 expressing B cells were harvested from the spleens of immunized mice shown to have elevated anti-hANGPTL3 antibody titers and were fused with mouse myeloma cells to form hybridomas. The hybridomas were screened and selected to identify cell lines expressing hANGPTL3-specific antibodies using assays as described below. The assays identified several cell lines that produced chimeric anti-hANGPTL3 antibodies, e.g., H1M896N.

Human ANGPTL3-specific antibodies were also isolated directly from antigen-immunized B cells without fusion to myeloma cells, as described in U.S. 2007/0280945 A1. Heavy and light chain variable regions were cloned to generate fully human anti-hANGPTL3 antibodies of IgG4 isotype designated as H4H1248P, H4H1250P, H4H1263S, H4H1268S, H4H1276S, H4H1279P, H4H1282P, H4H1292P, H4H1295P and H4H1296P. Stable recombinant antibody-expressing CHO cell lines were established.

### Example 2. Variable Gene Utilization Analysis

To analyze the structure of antibodies produced, the nucleic acids encoding antibody variable regions were cloned and sequenced. From the nucleic acid sequence and predicted amino acid sequence of the antibodies, gene usage was identified for each Heavy Chain Variable Region (HCVR) and Light Chain Variable Region (LCVR). Table 1 shows the gene usage for selected antibodies in accordance with the invention.

TABLE 1

| | |
|---|---|
| HCVR | LCVR |

| Antibody | V_{H} | D_{H} | J_{H} | V_{K} | J_{K} |
|---|---|---|---|---|---|
| H4H1248P | 3-30 | 1-26 | 6 | 1-12 | 3 |
| H4H1250P | 3-30 | 1-7 | 6 | 1-5 | 1 |
| H4H1263S | 3-30 | 3-10 | 6 | 1-12 | 3 |
| H4H1268S | 6-1 | 6-6 | 4 | 1-5 | 1 |
| H4H1276S | 3-43 | 3-3 | 3 | 1-5 | 2 |
| H4H1279P | 3-11 | 1-1 | 4 | 1-39 | 4 |
| H4H1282P | 1-18 | 3-10 | 4 | 1-9 | 4 |
| H4H1292P | 3-11 | 1-1 | 4 | 1-39 | 4 |
| H4H1295P | 1-18 | 6-25 | 4 | 2-30 | 2 |
| H4H1296P | 3-11 | 1-1 | 4 | 1-39 | 4 |
| H1M896N | 3-23 | 3-10 | 4 | 1-5 | 1 |

Table 2 shows the heavy and light chain variable region amino acid sequence pairs of selected anti-hANGPTL3 antibodies and their corresponding antibody identifiers. The N, P and S designations refer to antibodies having heavy and light chains with identical CDR sequences but with sequence variations in regions that fall outside of the CDR sequences (i.e., in the framework regions). Thus, N, P and S variants of a particular antibody have identical CDR sequences within their heavy and light chain variable regions but contain modifications within the framework regions.

TABLE 2

| | HCVR/LCVR |
|---|---|
| Name | SEQ ID NOs |
| H4H1248P | 2/10 |
| H4H1250P | 18/26 |
| H4H1263S | 34/42 |
| H4H1268S | 50/58 |
| H4H1276S | 66/74 |
| H1M896N | 180/188 |
| H4H1279P | 82/90 |
| H4H1282P | 98/106 |
| H4H1292P | 114/122 |
| H4H1295P | 130/138 |
| H4H1296P | 146/154 |
| — | — |

### Example 3. Kinetic Parameters of Anti-HANGPTL3 Antibodies Binding to ANGPTL3

All kinetic binding experiments were performed at 25° C. or 37° C. on a BIACORE™ T200 label-free molecular interaction instrument (GE Healthcare) using a CM5 sensor chip. Briefly, an antigen capture surface was generated by covalently coupling either an anti-mouse IgG-specific antibody (anti-mFc; GE Healthcare; catalog #BR-1008-38) or an anti-pentahistidine-specific antibody (Qiagen; catalog #34660) to the surface of a CM5 sensor chip using a standard amine coupling method. Using HBS-EP (10 mM HEPES, 150 mM NaCl, 3 mM EDTA, 0.05% Surfactant P20, pH 7.4) or PBSP (10 mM sodium phosphate, 2.7 mM KCl, 137 mM NaCl, 0.025% Surfactant P20, pH 7.2 or 5.75) as the running buffer, human and species variants of ANGPTL3 with oligohistidine tags were captured on the anti-penta-histidine coupled surface until a binding response of 4.4-46.5 RUs was achieved. The captured recombinant proteins were: full-length mature human ANGPTL3 (i.e., amino acid residues 17-460 of SEQ ID NO:161) with a C-terminal decahistidine tag (SEQ ID NO: 196) [hANGPTL3(17-460)-His; R&D Systems, MN; catalog #3829-AN], N-terminal coiled-coil domain of hANGPTL3 (i.e., amino acid residues 17-170 of SEQ ID NO:161) containing a C-terminal hexahistidine tag (SEQ ID NO: 195) [hANGPTL3(17-170)-His], N-terminal coiled-coil domain of ANGPTL3 from Macaca fascicularis [i.e., amino acid residues 17-170 of SEQ ID NO:177 (a partial sequence of Macaca fascicularis ANGPTL3)] containing a myc-myc-hexahistidine tag [MfANGPTL3(17-170)-mmH; SEQ ID NO:167], full-length mature ANGPTL3 from Mus musculus (i.e., amino acid residues 17-455 of SEQ ID NO: 163) with a C-terminal decahistidine tag (SEQ ID NO: 196) [mANGPTL3(17-455)-His; R&D Systems, MN; catalog #136-AN], N-terminal coiled-coil domain of ANGPTL3 from Mus musculus (i.e., amino acid residues 17-240 of SEQ ID NO:163) containing a hexahistidine tag (SEQ ID NO: 195) [mANGPTL3 (17-240)-His; SEQ ID NO:166], and N-terminal coiled-coil domain of ANGPTL3 from Rattus norvegicus (i.e., amino acid residues 17-240 of SEQ ID NO:175) containing a myc-myc-hexahistidine tag [rANGPTL3(17-240)-mmH; SEQ ID NO:176]. In addition, the N-terminal coiled-coil domain of hANGPTL3 (i.e., amino acid residues 17-169 of SEQ ID NO:161) containing a C-terminal mouse Fc fusion [hANGPTL3(17-169)-mFc; SEQ ID NO:165] was captured on the anti-mFc coupled surface until a binding response of 24.8±1.5 RUs was achieved. To measure association and dissociation rates for formation of the antibody/antigen complex, a single (Tables 3 and 7) or multiple (Tables 4-6) concentrations of antibody were injected across the captured protein surface at a flow rate of 50 μl/minute for 3 minutes and dissociation of the complex was monitored for 20 minutes. Binding data were processed and fitted to a 1:1 binding model with mass transport using Scrubber version 2.0a (BioLogic Software). The kinetic half-lives (t_{1/2}) were calculated from the dissociation rate constant, kd.

Table 3 shows the binding of various anti-ANGPTL3 antibodies to hANGPTL3 at 25° C., pH 7.4, in HBS-EP buffer.

TABLE 3

| | | ka | kd | K_{D} | t_{1/2} |
|---|---|---|---|---|---|
| Ab Clones | Protein | (M⁻¹s⁻¹) | (s⁻¹) | (nM) | (min) |
| H4H1248P | hANGPTL3(17- | 4.57E+05 | 2.72E−03 | 5.95 | 4 |
| | 169)-mFc | | | | |
| | hANGPTL3(17- | 4.40E+05 | 2.47E−03 | 5.62 | 5 |
| | 460)-His | | | | |
| H4H1250P | hANGPTL3(17- | 1.25E+06 | 6.51E−04 | 0.519 | 18 |
| | 169)-mFc | | | | |
| | hANGPTL3(17- | 9.04E+05 | 6.57E−04 | 0.726 | 18 |
| | 460)-His | | | | |
| H4H1263S | hANGPTL3(17- | 6.77E+05 | 4.22E−03 | 6.23 | 3 |
| | 169)-mFc | | | | |
| | hANGPTL3(17- | 5.08E+05 | 1.26E−03 | 2.47 | 9 |
| | 460)-His | | | | |
| H4H1268S | hANGPTL3(17- | 1.16E+06 | 8.35E−04 | 0.721 | 14 |
| | 169)-mFc | | | | |
| | hANGPTL3(17- | 1.29E+06 | 1.89E−03 | 1.47 | 6 |
| | 460)-His | | | | |
| H4H1276S | hANGPTL3(17- | 5.82E+05 | 3.83E−04 | 0.659 | 30 |
| | 169)-mFc | | | | |
| | hANGPTL3(17- | 3.44E+05 | 4.64E−04 | 1.35 | 25 |
| | 460)-His | | | | |
| H4H1279P | hANGPTL3(17- | 6.58E+05 | 5.53E−06 | 0.00841 | 2088 |
| | 169)-mFc | | | | |
| | hANGPTL3(17- | 2.88E+05 | 1.14E−04 | 0.394 | 102 |
| | 460)-His | | | | |
| H4H1282P | hANGPTL3(17- | 1.28E+06 | 5.92E−05 | 0.0463 | 195 |
| | 169)-mFc | | | | |
| | hANGPTL3(17- | 9.57E+05 | 9.26E−05 | 0.0968 | 125 |
| | 460)-His | | | | |
| H4H1292P | hANGPTL3(17- | 6.86E+05 | 1.77E−04 | 0.257 | 65 |
| | 169)-mFc | | | | |
| | hANGPTL3(17- | 3.41E+05 | 2.48E−04 | 0.727 | 47 |
| | 460)-His | | | | |
| H4H1295P | hANGPTL3(17- | 3.52E+05 | 7.95E−05 | 0.226 | 145 |
| | 169)-mFc | | | | |
| | hANGPTL3(17- | 3.73E+05 | 7.35E−05 | 0.197 | 157 |
| | 460)-His | | | | |
| H4H1296P | hANGPTL3(17- | 6.41E+05 | 3.92E−05 | 0.0611 | 295 |
| | 169)-mFc | | | | |
| | hANGPTL3(17- | 3.01E+05 | 4.12E−05 | 0.137 | 280 |
| | 460)-His | | | | |

As shown in Table 3, the anti-hANGPLT3 antibodies bound to the full-length protein with a C-terminal decahistidine tag (SEQ ID NO: 196) [hANGPTL3(17-460)-His] with calculated equilibrium dissociation constants (K_{D}=kd/ka) ranging from 96.8 pM to 5.62 nM and to the N-terminal coiled-coil domain with a C-terminal Fc fusion [hANGPTL3(17-169)-mFc] with K_{D}s ranging from 8.41 pM to 6.23 nM.

Tables 4 and 5 show the cross-species binding of H4H1276S to ANGPTL3 at 25° C. and 37° C., respectively, at pH 7.4, in HBS-EP buffer. Table 6 shows the binding of H4H1276S to human and cynomolgus ANGPTL3, at 25° C. or 37° C., at pH 5.75 or pH 7.2, in PBSP buffer.

TABLE 4

| | | 25° C. | | | |
|---|---|---|---|---|---|
| | | ka | kd | K_{D} | t_{1/2} |
| Ab Clone | Protein | (M⁻¹s⁻¹) | (s⁻¹) | (nM) | (min) |
| H4H1276S | hANGPTL3(17- | 9.73E+05 | 9.12E−04 | 0.938 | 12.7 |
| | 170)-His | | | | |
| | hANGPTL3(17- | 5.88E+05 | 2.89E−04 | 0.491 | 40.0 |
| | 460)-His | | | | |
| | MfANGPTL3(17- | 1.35E+06 | 5.35E−04 | 0.396 | 21.6 |
| | 170)-mmH | | | | |
| | mANGPTL3(17- | 6.70E+05 | 3.07E−04 | 0.458 | 37.6 |
| | 240)-His | | | | |
| | mANGPTL3(17- | 1.29E+06 | 3.46E−04 | 0.268 | 33.4 |
| | 455)-His | | | | |
| | rANGPTL3(17- | 1.35E+06 | 7.18E−04 | 0.530 | 16.1 |
| | 240)-mmH | | | | |

TABLE 5

| | | 37° C. | | | |
|---|---|---|---|---|---|
| Ab Clone | Protein | ka (M⁻¹s⁻¹) | kd (s⁻¹) | K_{D} (nM) | t_{1/2} (min) |
| H4H1276S | hANGPTL3(17-170)-His | 1.59E+06 | 2.41E−03 | 1.52 | 4.8 |
| | hANGPTL3(17-460)-His | 6.32E+05 | 8.12E−04 | 1.29 | 14.2 |
| | MfANGPTL3(17-170)-mmH | 1.87E+06 | 1.17E−03 | 0.625 | 9.9 |
| | mANGPTL3(17-240)-His | 8.19E+05 | 9.64E−04 | 1.18 | 12.0 |
| | mANGPTL3(17-455)-His | 1.94E+06 | 7.91E−04 | 0.408 | 14.6 |
| | rANGPTL3(17-240)-mmH | 2.05E+06 | 1.93E−03 | 0.940 | 6.0 |

TABLE 6

| Ab Clone | Protein | ka (M⁻¹s⁻¹) | kd (s⁻¹) | K_{D} (nM) | t_{1/2} (min) |
|---|---|---|---|---|---|
| H4H1276S | hANGPTL3(17-170)-His | 1.00E+06 | 1.10E−03 | 1.09 | 10.5 |
| pH 7.2 | hANGPTL3(17-460)-His | 5.99E+05 | 4.02E−04 | 0.670 | 28.8 |
| 25° C. | MfANGPTL3(17-170)-mmH | 1.45E+06 | 5.38E−04 | 0.370 | 21.5 |
| H4H1276S | hANGPTL3(17-170)-His | 2.80E+05 | 6.72E−03 | 24.0 | 1.7 |
| pH 5.75 | hANGPTL3(17-460)-His | 7.32E+04 | 4.94E−03 | 67.5 | 2.3 |
| 25° C. | MfANGPTL3(17-170)-mmH | 2.06E+05 | 4.32E−03 | 21.0 | 2.7 |
| H4H1276S | hANGPTL3(17-170)-His | 1.57E+06 | 2.73E−03 | 1.74 | 4.2 |
| pH 7.2 | hANGPTL3(17-460)-His | 6.67E+05 | 1.18E−03 | 1.76 | 9.8 |
| 37° C. | MfANGPTL3(17-170)-mmH | 1.94E+06 | 1.36E−03 | 0.700 | 8.5 |
| H4H1276S | hANGPTL3(17-170)-His | 1.22E+06 | 3.24E−02 | 26.7 | 0.4 |
| pH 5.75 | hANGPTL3(17-460)-His | 4.71E+04 | 1.07E−02 | 227 | 1.1 |
| 37° C. | MfANGPTL3(17-170)-mmH | 2.78E+05 | 5.21E−03 | 18.8 | 2.2 |

As shown in Tables 4-6, antibody H4H1276S exhibited binding to ANGPTL3 from monkey, mouse, and rat with binding affinities and kinetic constants similar to those for binding to human ANGPTL3.

Table 7 shows the binding of selected anti-ANGPTL3 antibodies to hANGPTL3 and mANGPTL3 at 37° C., pH 7.4, in HBS-EP buffer. NB: Not bound.

TABLE 7

| Ab Clones | Protein | ka (M⁻¹s⁻¹) | kd (s⁻¹) | K_{D} (nM) | t_{1/2} (min) |
|---|---|---|---|---|---|
| H1M896N | hANGPTL3(17-460)-His | 3.36E+06 | 5.30E−04 | 1.58E−10 | 22 |
| | mANGPTL3(17-455)-His | 3.62E+06 | 2.47E−03 | 6.82E−10 | 5 |
| H4H1248P | hANGPTL3(17-460)-His | 1.96E+06 | 1.93E−03 | 9.86E−10 | 6 |
| | mANGPTL3(17-455)-His | NB | NB | NB | NB |
| H4H1250P | hANGPTL3(17-460)-His | 3.50E+06 | 1.13E−03 | 3.24E−10 | 10 |
| | mANGPTL3(17-455)-His | 3.18E+06 | 1.55E−03 | 4.86E−10 | 7 |
| H4H1263S | hANGPTL3(17-460)-His | 4.74E+06 | 1.81E−03 | 3.81E−10 | 6 |
| | mANGPTL3(17-455)-His | NB | NB | NB | NB |
| H4H1279P | hANGPTL3(17-460)-His | 4.74E+06 | 1.81E−03 | 3.81E−10 | 6 |
| | mANGPTL3(17-455)-His | 2.15E+06 | 3.59E−04 | 1.67E−10 | 32 |
| H4H1292P | hANGPTL3(17-460)-His | 1.89E+06 | 1.94E−03 | 1.02E−09 | 6 |
| | mANGPTL3(17-455)-His | 3.92E+06 | 1.49E−03 | 3.80E−10 | 8 |

As shown in Table 7, the anti-hANGPLT3 antibodies bound to the full-length protein with a C-terminal decahistidine tag (SEQ ID NO: 196) [hANGPTL3(17-460)-His] at pH 7.4 and 37° C. with calculated equilibrium dissociation constants (K_{D}=kd/ka) ranging from 158 pM to 1.02 nM and to mouse ANGPTL3 [mANGPTL3(17-455)-His] with K_{D}s ranging from 167 pM to 682 pM, except for H4H1248P and H4H1263S, which did not show detectable binding to mANGPTL3. Also shown are the kinetic half-lives (t_{1/2}).

### Example 4. Biacore Cross-Competition Study for Anti-ANGPTL3 Antibodies

Cross competition experiments were performed at 25° C. on a Biacore essentially as described in Example 3 above. Briefly, using HBS-EP as the running buffer, full-length hANGPTL3 (i.e., amino acid residues 17-460 of SEQ ID NO: 161) with a C-terminal decahistidine tag (SEQ ID NO: 196) [hANGPTL3 (17-460)-His; R&D Systems, MN; catalog #3829-AN] was captured on the anti-penta-histidine coupled surface until a binding response of 64 RUs was achieved. To determine whether two antibodies could bind simultaneously to the captured ANGPTL3, antibody pairs were injected sequentially, each at 167 nM at a flow rate of 4 μl/minute for 15 minutes, over the surface, and the maximum binding response signal (RU) was measured for each binding event. The results are shown in Table 8 with binding response for the first antibody (mAb1), followed by binding response of the second antibody (mAb2) on the ANGPTL3 surface pre-loaded with the first antibody. Numbers in bold indicate that the antibody pairs are able to bind to hANGPTL3simultaneously. Numbers in italics indicate that that the antibody pairs are able to bind to hANGPTL3 simultaneously when added sequentially in one direction but not the other. Brackets indicate self-self competition.

TABLE 8

| | hANGPTL3(17- | 25 μg/ml | | | | |
|---|---|---|---|---|---|---|
| | 460)-His | mAb1 binding | 25 μg/ml mAb2 binding response (RU) | | | |
| Ab Clones | captured (RU) | response (RU) | H1M896N | H4H1250P | H4H1279P | H4H1292P |
| H1M896N | 64 ± 4 | 171 ± 1.8 | [−4] | −4 | 47 | 50 |
| H4H1250P | | 210 ± 8.3 | −4 | [13] | 9 | 8 |
| H4H1279P | | 45 ± 1.8 | 186 | 213 | [−3] | −1 |
| H4H1292P | | 48 ± 0.7 | 182 | 220 | −3 | [−2] |
| Negative | | 81 ± 1.8 | 149 | 187 | 47 | 48 |
| Control | | | | | | |

As shown in Table 8, antibody pairs H1M896N/H4H1279P and H1M896N/H4H1292P were able to bind simultaneously to immobilized ANGPTL3, regardless of the order of addition of the antibodies. H4H1250P bound to ANGPTL3 pre-bound with H4H1279P; however, when the order of antibody addition was reversed, H4H1279P exhibited a binding signal approximately 24% of the expected maximal response after ANGPTL3 was pre-bound with H4H1250P. Similarly, H4H1250P bound to ANGPTL3 pre-bound with H4H1292P; however, when the order of antibody addition was reversed, H4H1292P exhibited a binding signal approximately 20% of the expected maximal response after ANGPTL3 was pre-bound with H4H1250P.

### Example 5. Anti-HANGPTL3 Antibody Binding to ANGPTL3 N-terminal Coiled-Coil Peptides

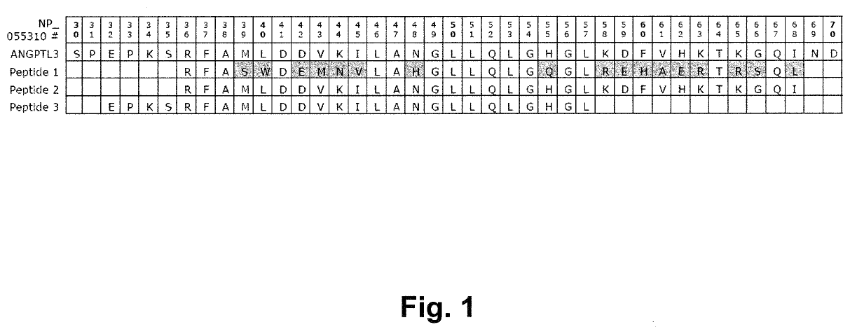

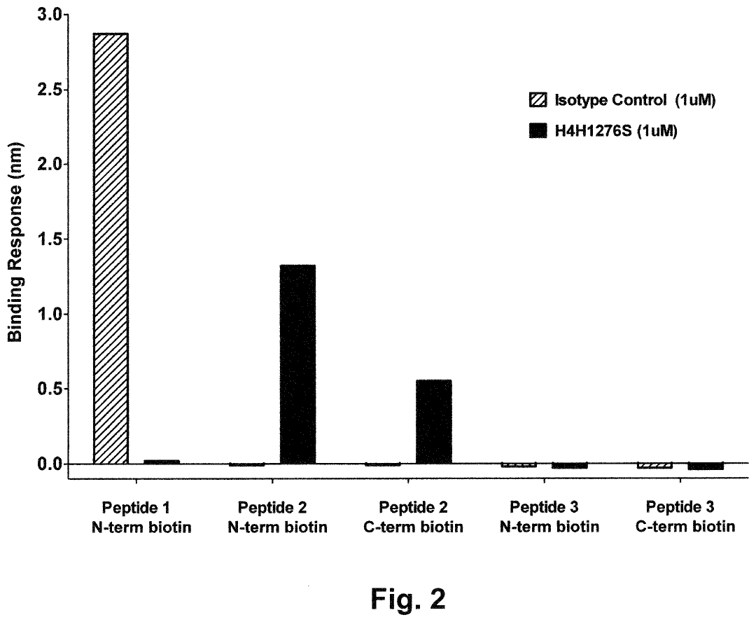

To assess the binding of the anti-ANGPTL3 antibody H4H1276S to peptides derived from the N-terminal coiled-coil region of ANGPTL3, a label-free biosensor binding assay was performed using OCTET® RED system (FortéBio, Inc.). For immobilization onto the sensor, peptides were labeled with either an N-terminal biotin tag [separated by a flexible linker, amino acids “AGSSPGG” (SEQ ID NO: 171), for Peptide 1 and Peptide 2; and amino acids “GGGGS” (SEQ ID NO:172) for Peptide 3], or a C-terminal biotin tag [separated by a flexible linker, amino acids “GPSSGAPPPK” (SEQ ID NO: 173), for Peptide 1 and Peptide 2; and amino acids “GGGGSK” (SEQ ID NO:174) for Peptide 3]. The peptide sequences tested were: A negative control peptide, N-terminal biotin tagged Peptide1 (SEQ ID NO:168; residues Arg34 to Leu66 of human ANTGPTL4 of SEQ ID NO: 164); and peptides derived from the N-terminal coiled-coil region of ANGPTL3, i.e., N-terminal biotin tagged Peptide 2 (SEQ ID NO: 169; residues Arg36 to Leu68 of hANGPTL3 of SEQ ID NO: 161); C-terminal biotin tagged Peptide 2; N-terminal biotin tagged Peptide 3 (SEQ ID NO:170;corresponds to residues Glu32 to Leu57 of hANGPTL3 of SEQ ID NO: 161); and C-terminal biotin tagged Peptide 3. Peptide sequences are also shown in FIG. 1. Streptavidin-coated biosensor tips were coated with the biotinylated peptides resulting in 1.22-2.26 nm of binding response units depending on the peptide. The peptide-coated biosensor tips were then dipped into wells containing 1 μM of either anti-ANGPTL3 antibody H4H1276S or an isotype-matched negative control antibody, and binding was monitored for 2.5 minutes. The binding response of H4H1276S and the isotype control antibody to each of the peptides is summarized in FIG. 2. It was observed that H4H1276S binds to the ANGPTL3 linear sequence defined by Peptide 2 but not the overlapping but distinct sequence defined by Peptide 3 (see also FIG. 1). The isotype control antibody also served as a positive control for loading of Peptide 1 (i.e., hANGPTL4 peptide) onto the biosensor, because this isotype control antibody specifically recognizes hANGPTL4. As shown in FIG. 2, the binding of the control antibody to Peptide 1 confirmed that Peptide 1 was present on the sensor surface and so were the other peptides.

### Example 6. Inhibition of hANGPTL3 by Anti-hANGPTL3 Antibodies in LPL Bioassays

Lipoprotein Lipase (LPL) plays a critical role in lipid metabolism in humans. LPL catalyzes hydrolysis of triglycerides and releases fatty acids to be metabolized. ANGPTL3 inhibits LPL activity leading to increased level of lipids (Oike, et al., 2005, Trends in Molecular Medicine 11(10):473-479). The N-terminal coiled-coil region of ANGPTL3 inhibits LPL when expressed without the C-terminal fibrinogen region and therefore appears to confer its inhibitory function. A cell-free bioassay was developed to determine the ability of anti-ANGPTL3 antibodies to inhibit ANGPTL3-induced decrease in LPL activity.

Inhibition of hANGPTL3 activity by anti-ANGPTL3 antibodies was determined using the CONFLUOLIP™ Continuous Fluorometric Lipase Test (Progen, Germany) using three hANGPTL3proteins: full-length mature hANGPTL3 (i.e., amino acid residues 17-460 of SEQ ID NO: 161) with a C-terminal decahistidine tag (SEQ ID NO: 196) [hANGPTL3 (17-460)-His; R&D Systems, MN; catalog #3829-AN], the N-terminal coiled-coil region (i.e., amino acid residues 17-169 of SEQ ID NO: 161) with a C-terminal mouse Fc fusion [hANGPTL3 (17-169)-mFc; SEQ ID NO: 165], and the N-terminal coiled-coil domain of hANGPTL3 (i.e., amino acid residues 17-170 of SEQ ID NO: 161) containing a C-terminal hexahistidine tag (SEQ ID NO: 195) [hANGPTL3 (17-170)-His].

Briefly, bovine LPL (final concentration of 2 nM), human ApoCII (a cofactor of LPL, final concentration of 0.23 μM), and BSA (final concentration of 2 mg/mL) in PBS were premixed. The hANGPTL3 recombinant proteins were added to the Apo/LPL mixture (final concentrations of 80-100 nM). The Apo/LPL/ANGPTL3 protein mixtures were then added together with serially diluted anti-hANGPTL3 antibodies and incubated at room temperature for 30 minutes. Following the incubation, 100 μl of reconstituted lipase substrate, 1-trinitrophenyl-amino-dodecanoyl-2-pyrendecanoyl-3-0-hexadecyl-sn-glycerol (LS-A, Progen), was added to 25 μl of the antibody mixture to a 96-well assay plate and incubated at 37° C. for two hours. Fluorescence was then measured at 342 nm/400 nm (excitation/emission) using a FLEXSTATION® 3 Microplate Reader (Molecular Devices, CA). Fluorescence is directly proportional to LPL activity.

Antibody H4H1276S exhibited inhibition of hANGPTL3′s inhibitory activity against LPL. A full dose-response using the hANGPTL3 protein in the LPL assay was first performed to determine the ANGPTL3 EC₅₀ for each experiment, and IC₅₀ determinations for the antibody were then performed using constant concentrations of ANGPTL3 protein, as shown in Table 8. The antibody concentrations required for 50% maximum inhibition (IC₅₀) was determined to be 9.6 nM for 80 nM hANGPTL3 (17-460)-His, 2.9 nM for 100 nM hANGPTL3 (17-170)-His and 21 nM for 80 nM hANGPTL3 (17-169)-mFc, respectively. Antibody concentrations ranged from 0 to 300 nM for testing human ANGPTL3 proteins.

Similarly, H4H1276S was tested in the LPL bioassay for its ability to inhibit cross-species orthologs: the cynomolgus monkey N-terminal region (amino acid residues 17-170 of SEQ ID NO: 177) expressed with an C-terminal myc-myc-hexa-histidine tag [MfANGPTL3 (17-170)-mmH; SEQ ID NO: 167], the mouse ortholog N-terminal region amino acid residues 17-240 of SEQ ID NO: 163 with a C-terminal hexa-histidine tag [mANGPTL3 (17-240)-His; SEQ ID NO: 166], and full-length mature ANGPTL3 from Mus musculus (i.e., amino acid residues 17-455 of SEQ ID NO: 163) with a C-terminal decahistidine tag (SEQ ID NO: 196) [mANGPTL3 (17-455)-His; R&D Systems, MN; catalog #136-AN]. IC50s were determined to be 10 nM for 500 nM constant MfANGPTL3(17-170)-mmH, 14 nM for 80 nM constant mANGPTL3 (17-455)-His, and 31 nM for 500 nM constant mANGPTL3(17-240)-His. Antibody concentrations ranged from 0 to 600 nM for testing monkey and mouse ANGPTL3 proteins. The results are summarized in Table 9.

Antibodies against the N-terminal region of the homologous protein ANGPTL4 have also been shown to block the inhibitory function of ANGPTL4 on LPL (Lee, et al., 2009, J. Biol. Chem. 284:13735-13745). Therefore, to evaluate possible cross-reactivity to ANGPTL4, the inhibitory anti-ANGPTL3 antibody H4H1276S was also tested against human ANGPTL4 in the LPL lipase assay, conducted as described above for the ANGPTL3 proteins. A recombinant form of the coiled-coil region of human ANGPTL4 (residues 26-148 of SEQ ID NO:164) with a C-terminal mouse IgG2a Fc fusion [hANGPTL4 (26-148)-mFc, SEQ ID NO:178] exhibited an EC50 in the LPL assay of 0.2 nM (Table 9). H4H1276S, tested through a concentration range of 0-600 nM, did not block this inhibition (NB: Not bound; in Table 9).

TABLE 9

| | | Human | Human | Human | Monkey | Mouse | Mouse | Human |
|---|---|---|---|---|---|---|---|---|
| | | ANGPTL3 | ANGPTL3 | ANGPTL3 | ANGPTL3 | ANGPTL3 | ANGPTL3 | ANGPTL4 |
| | | (17-460)-His | (17-170)-His | (17-169)-mFc | (17-170)-mmH | (17-455)-His | (17-240)-His | (26-148)-mFc |
| EC₅₀ (nM) | | 50 | 91 | 16 | 625 | 33 | 199 | 0.2 |
| Constant | | 80 | 100 | 80 | 500 | 80 | 500 | 2 |
| ANGPTL3 or | | | | | | | | |
| 4 (nM) | | | | | | | | |
| IC₅₀ | H4H 1276S | 9.6 | 2.9 | 21 | 10 | 14 | 31 | NB |
| (nM) | IgG4 cont. | NB | NB | NB | NB | NB | NB | NB |

As shown above, H4H1276S inhibited human ANGPTL3 (full-length and N-terminal), monkey ANGPTL3 (N-terminal) protein and mouse ANGPTL3 (full-length and N-terminal) activity at comparable degrees with an IC₅₀ range of about 3-31 nM.

A subset of antibodies were also tested to determine if combinations of two ANGPTL3 non-blocking antibodies added simultaneously could block the LPL inhibitory activity of ANGPTL3. Pairs of antibodies were tested for inhibiting the N-terminal domains of both human and mouse ANGPTL3, i.e., hANGPTL3 (17-169)-mFc and mANGPTL3 (17-240)-His, respectively. For this assay, the ANGPTL3 proteins exhibited IC50 values for blocking LPL of 47 nM [for hANGPTL3(17-169)-mFc] and 341 nM [for mANGPTL3 (17-240)-His]. The following pairs, when added at final concentrations for each antibody of at least 200nM, did not block the inhibition of LPL by either hANGPTL3 (17-169)-mFc at 80nM or mANGPTL3 (17-240)-His at 500 nM: H1M896N+H4H1279P; H4H1250P+H4H1279P; H4H1248P+H4H1292P; and H4H1263S+H4H1292P. In this same assay, H4H1276S alone blocked these same constant concentrations of human and mouse ANGPTL3 with IC50s of 33 nM and 64 nM, respectively.

Example 7.1. In Vivo Effect of Anti-ANGPTL3 Antibody on Serum Lipid Levels

The effect of the anti-hANGPTL3 antibody H4H1726S on serum lipid levels was determined in C57BI/6 mice. Mice were pre-bled 7 days before the experiment and put into groups of six mice each for each antibody dose tested. Antibodies were administered at 5 mg/kg (H4H1726S) and 10 mg/kg [H4H1726S and isotype-matched hlgG4 (S108P) control with irrelevant specificity] dose levels by subcutaneous injection on day 0 of the study. Mice were bled after 4hours of fasting at days 1, 4, 7 and 12 after antibody injections and serum lipid levels (triglycerides, total cholesterol, non-HDL cholesterol, LDL cholesterol and HDL cholesterol) were determined in the serum by an ADVIA® 1800 Chemistry System (Siemens). Averages were calculated for each of the time points for each antibody. Results, expressed as (mean±SEM) of serum lipid concentration, are shown in Tables 10-14.

TABLE 10

| | Serum triglycerides (mg/dL) | | | | | |
|---|---|---|---|---|---|---|
| | Control Ab | | H4H1276S | | H4H1276S | |
| Days after | (10 mg/kg) | | (5 mg/kg) | | (10 mg/kg) | |
| injection | Mean | SEM | Mean | SEM | Mean | SEM |
| −7 | 87.83 | 6.18 | 89.83 | 3.65 | 87.17 | 5.062 |
| 1 | 123.16 | 7.02 | 68.00 | 2.84 | 53.83 | 2.52 |
| 4 | 99.66 | 10.15 | 62.16 | 5.82 | 50.67 | 3.51 |
| 7 | 99.83 | 4.57 | 55.83 | 4.95 | 39.67 | 2.55 |
| 12 | 82.00 | 5.75 | 76.83 | 10.56 | 53.00 | 6.51 |

TABLE 11

| | Total cholesterol (mg/dL) | | | | | |
|---|---|---|---|---|---|---|
| | Control Ab | | H4H1276S | | H4H1276S | |
| Days after | (10 mg/kg) | | (5 mg/kg) | | (10 mg/kg) | |
| injection | Mean | SEM | Mean | SEM | Mean | SEM |
| −7 | 82.50 | 2.11 | 80.33 | 1.15 | 81.33 | 2.14 |
| 1 | 87.83 | 1.87 | 71.50 | 5.48 | 63.67 | 3.38 |
| 4 | 75.00 | 2.58 | 59.50 | 3.51 | 51.00 | 2.98 |
| 7 | 83.50 | 1.77 | 67.00 | 1.79 | 61.33 | 2.33 |
| 12 | 87.83 | 1.82 | 83.00 | 4.30 | 69.33 | 3.22 |

TABLE 12

| | Non-HDL cholesterol (mg/dL) | | | | | |
|---|---|---|---|---|---|---|
| | Control Ab | | H4H1276S | | H4H1276S | |
| Days after | (10 mg/kg) | | (5 mg/kg) | | (10 mg/kg) | |
| injection | Mean | SEM | Mean | SEM | Mean | SEM |
| −7 | 41.18 | 0.75 | 38.78 | 0.81 | 40.23 | 1.18 |
| 1 | 42.18 | 0.55 | 35.75 | 3.05 | 32.70 | 1.94 |
| 4 | 36.40 | 1.04 | 29.63 | 2.16 | 27.55 | 1.78 |
| 7 | 40.82 | 0.75 | 34.67 | 1.83 | 32.02 | 1.68 |
| 12 | 41.72 | 0.87 | 39.85 | 2.21 | 35.13 | 1.47 |

TABLE 13

| | LDL cholesterol (mg/dL) | | | | | |
|---|---|---|---|---|---|---|
| | Control Ab | | H4H1276S | | H4H1276S | |
| Days after | (10 mg/kg) | | (5 mg/kg) | | (10 mg/kg) | |
| injection | Mean | SEM | Mean | SEM | Mean | SEM |
| −7 | 4.68 | 0.35 | 4.40 | 0.34 | 4.47 | 0.21 |
| 1 | 5.40 | 0.41 | 5.20 | 0.79 | 5.33 | 0.71 |
| 4 | 4.80 | 0.45 | 4.88 | 0.67 | 5.33 | 0.73 |
| 7 | 5.38 | 0.46 | 5.83 | 0.48 | 6.40 | 0.67 |
| 12 | 5.67 | 0.59 | 6.12 | 0.65 | 5.35 | 0.48 |

TABLE 14

| | HDL cholesterol (mg/dL) | | | | | |
|---|---|---|---|---|---|---|
| | Control Ab | | H4H1276S | | H4H1276S | |
| Days after | (10 mg/kg) | | (5 mg/kg) | | (10 mg/kg) | |
| injection | Mean | SEM | Mean | SEM | Mean | SEM |
| −7 | 41.32 | 1.57 | 41.55 | 0.90 | 41.10 | 1.37 |
| 1 | 45.65 | 1.85 | 35.75 | 2.54 | 30.97 | 2.13 |
| 4 | 38.60 | 2.26 | 29.87 | 1.62 | 23.45 | 1.66 |
| 7 | 42.68 | 1.81 | 32.33 | 1.25 | 29.32 | 1.72 |
| 12 | 46.12 | 1.94 | 43.15 | 2.52 | 34.20 | 1.99 |

Levels of circulating H4H1726S (Serum Ab) were also determined using a standard ELISA assay. Briefly, plates were coated with a goat anti-human Fc antibody (Sigma-Aldrich) to capture Serum Ab. Serum was then added to the plates and captured human antibody was detected by chemiluminescence using a horseradish peroxidase (HRP) conjugated goat anti-human IgG antibody (Sigma-Aldrich). Results, expressed as (mean±SEM) of are shown in Table 15. Control: Mice that received an isotype-matched Control Ab.

TABLE 15

| | Serum Ab (μg/mL) | | | | | |
|---|---|---|---|---|---|---|
| | Control Ab | | H4H1276S | | H4H1276S | |
| Days after | (10 mg/kg) | | (5 mg/kg) | | (10 mg/kg) | |
| injection | Mean | SEM | Mean | SEM | Mean | SEM |
| 1 | 65.00 | 8.05 | 36.38 | 7.57 | 126.23 | 9.96 |
| 4 | 59.16 | 4.94 | 29.91 | 4.32 | 86.28 | 6.77 |
| 7 | 58.23 | 6.02 | 30.86 | 5.11 | 54.24 | 8.96 |
| 12 | 41.35 | 9.76 | 5.48 | 1.79 | 39.04 | 7.08 |

Single administration of H4H1276S to C57BI/6 mice at 10 mg/kg led to ˜60% reduction in circulating triglycerides 7 days after the antibody administration (compared to isotype control). The administration of H4H1276S also led to a significant reduction in total cholesterol, non-HDL cholesterol and HDL cholesterol and had no effect on LDL cholesterol. A reduction in lipid levels was also observed, but less pronounced, at the 5 mg/kg compared to 10 mg/kg dose levels; e.g., serum triglycerides were reduced by 44% (compared to isotype control) 7 days after antibody administration.

### Example 7.2. In Vivo Effect of Anti-ANGPTL3 Antibodies on Serum Lipid Levels

The evaluation of the in vivo effects of anti-hANGPTL3 antibodies H4H1276S and comparator antibody 4.9.1 on serum lipid levels was conducted in C57BI/6 mice. Antibody 4.9.1was prepared based on the amino acid sequences of SEQ ID No: 24 (VH) and SEQID No: 32 (VL) as disclosed in US Patent Application Publication No. 2008/0177045 and as a mouse IgG1 isotype. Mice were pre-bled 7 days before the experiment and put into groups of six mice per group. Antibodies H4H1276S, 4.9.1, and isotype-matched negative controls (human lgG4 and mouse lgG1, respectively) with irrelevant specificity were administered at 10 mg/kg dose by subcutaneous injection on day 0 of the study. Mice were bled after 4 hours of fasting at days 1, 7, 11 and 20 after injection of antibodies, and serum lipids levels (triglycerides, total cholesterol, non-HDL cholesterol, LDL cholesterol and HDL cholesterol) were determined in the serum using an ADVIA® 1800 Chemistry System (Siemens). Average lipid concentrations were calculated for each of the time point for each antibody. Results, expressed as (mean±SEM) of serum lipid concentration, are shown in Tables 16-20.

TABLE 16

| | Serum triglycerides (mg/dL) | | | | | | | |
|---|---|---|---|---|---|---|---|---|
| Days after | Control (IgG4) | | H4H1276S | | Control (IgG1) | | 4.9.1 | |
| injection | Mean | SEM | Mean | SEM | Mean | SEM | Mean | SEM |
| −7 | 109.16 | 9.05 | 109.16 | 6.44 | 112.80 | 6.87 | 109.17 | 7.24 |
| 1 | 81.67 | 6.76 | 46.00 | 3.59 | 95.20 | 8.92 | 41.83 | 2.42 |
| 7 | 95.67 | 5.42 | 49.67 | 3.86 | 101.80 | 7.55 | 96.00 | 3.70 |
| 11 | 100.83 | 6.20 | 51.00 | 5.89 | 117.00 | 6.00 | 92.00 | 4.50 |
| 20 | 82.17 | 4.36 | 72.67 | 3.47 | 79.40 | 6.59 | 73.83 | 5.03 |

TABLE 17

| | Total cholesterol (mg/dL) | | | | | | | |
|---|---|---|---|---|---|---|---|---|
| Days after | Control (IgG4) | | H4H1276S | | Control (IgG1) | | 4.9.1 | |
| injection | Mean | SEM | Mean | SEM | Mean | SEM | Mean | SEM |
| −7 | 80.81 | 0.95 | 80.92 | 3.05 | 80.32 | 2.84 | 79.37 | 2.76 |
| 1 | 82.82 | 2.11 | 67.33 | 3.60 | 82.98 | 2.17 | 71.35 | 1.82 |
| 7 | 79.20 | 1.81 | 63.58 | 3.98 | 85.02 | 7.27 | 82.07 | 4.36 |
| 11 | 89.97 | 3.18 | 69.02 | 2.11 | 83.92 | 2.49 | 84.58 | 1.08 |
| 20 | 92.43 | 1.10 | 80.17 | 3.20 | 87.47 | 2.58 | 88.40 | 2.84 |

TABLE 18

| | Non-HDL cholesterol (mg/dL) | | | | | | | |
|---|---|---|---|---|---|---|---|---|
| Days after | Control (IgG4) | | H4H1276S | | Control (IgG1) | | 4.9.1 | |
| injection | Mean | SEM | Mean | SEM | Mean | SEM | Mean | SEM |
| −7 | 42.44 | 1.18 | 42.87 | 1.03 | 43.20 | 2.44 | 41.73 | 1.40 |
| 1 | 40.85 | 1.48 | 35.33 | 1.79 | 40.68 | 0.87 | 36.97 | 1.49 |
| 7 | 39.03 | 1.04 | 33.72 | 2.86 | 43.30 | 4.35 | 40.47 | 2.35 |
| 11 | 44.68 | 1.93 | 35.18 | 1.64 | 40.28 | 0.95 | 41.38 | 1.05 |
| 20 | 47.40 | 0.67 | 42.10 | 1.51 | 44.72 | 1.66 | 44.40 | 1.57 |

TABLE 19

| | LDL cholesterol (mg/dL) | | | | | | | |
|---|---|---|---|---|---|---|---|---|
| Days after | Control (IgG4) | | H4H1276S | | Control (IgG1) | | 4.9.1 | |
| injection | Mean | SEM | Mean | SEM | Mean | SEM | Mean | SEM |
| −7 | 3.93 | 0.07 | 4.20 | 0.26 | 4.38 | 0.26 | 4.20 | 0.18 |
| 1 | 3.95 | 0.28 | 4.25 | 0.37 | 3.92 | 0.17 | 4.62 | 0.37 |
| 7 | 3.75 | 0.14 | 5.25 | 1.08 | 5.76 | 1.61 | 4.57 | 0.73 |
| 11 | 5.05 | 0.26 | 5.47 | 0.23 | 4.88 | 0.27 | 4.78 | 0.23 |
| 20 | 5.72 | 0.34 | 4.95 | 0.32 | 4.97 | 0.28 | 5.65 | 0.46 |

TABLE 20

| | HDL cholesterol (mg/dL) | | | | | | | |
|---|---|---|---|---|---|---|---|---|
| Days after | Control (IgG4) | | H4H1276S | | Control (IgG1) | | 4.9.1 | |
| injection | Mean | SEM | Mean | SEM | Mean | SEM | Mean | SEM |
| −7 | 38.37 | 0.95 | 38.00 | 2.27 | 37.12 | 1.88 | 37.63 | 1.52 |
| 1 | 41.97 | 1.32 | 32.00 | 1.89 | 42.30 | 2.09 | 34.38 | 0.85 |
| 7 | 40.17 | 0.93 | 29.87 | 1.23 | 41.72 | 2.97 | 41.60 | 2.47 |
| 11 | 45.28 | 1.80 | 33.83 | 1.15 | 43.64 | 1.70 | 43.20 | 1.57 |
| 20 | 45.03 | 0.75 | 38.07 | 1.79 | 42.75 | 1.69 | 44.00 | 1.83 |

A single 10 mg/kg dose of H4H1276S in C57BI/6 mice resulted in reduction of plasma triglyceride levels compared to isotype control on days 1, 7, 11 and 20 after antibody injection; and this effect was more sustained compared to a single treatment at the same dose level with the comparator 4.9.1 (Table 16). Administration of H4H1276S also led to a reduction in total cholesterol (Table 17) and HDL cholesterol (Table 20) in C57BI/6 mice.

### Example 8. In Vivo Effect of H4H1276S on Serum Lipid Levels in Hyperlipidemic ApoE^{-/-} Mice

The effect of anti-hANGPTL3 antibody H4H1276S on serum lipids levels was determined in apoE^{-/-} mice. These mice are hyperlipidemic with the majority of their circulating cholesterol found in the form of VLDL and LDL. Mice were pre-bled 7 days before the experiment and put into groups of six mice per group. The antibodies, H4H1276S and an isotype-matched (hlgG4) control with irrelevant specificity, were administered at 10 mg/kg dose by subcutaneous injection on day 0 of the study. Mice were bled after 4 hours of fasting at days 1, 4, 7 and 11 after injection of antibodies; and serum lipids levels (triglycerides, total cholesterol, non-HDL cholesterol, LDL cholesterol and HDL cholesterol) were determined in the serum using an ADVIA® 1800 Chemistry System (Siemens). Average lipid concentrations were calculated for each of the time points for each antibody-treated group. Results, expressed as (mean±SEM) of serum lipid concentration, are shown in Tables 21-25.

TABLE 21

| | Serum triglycerides (mg/dL) | | | |
|---|---|---|---|---|
| Days after | Control (hIgG4) | | H4H1276S | |
| injection | Mean | SEM | Mean | SEM |
| −7 | 134.17 | 11.81 | 141.67 | 17.14 |
| 1 | 156.33 | 19.06 | 61.33 | 3.66 |
| 4 | 181.00 | 7.70 | 70.50 | 4.46 |
| 7 | 190.67 | 27.65 | 52.50 | 6.22 |
| 11 | 170.00 | 28.85 | 133.00 | 13.56 |

TABLE 22

| | Total cholesterol (mg/dL) | | | |
|---|---|---|---|---|
| Days after | Control (hIgG4) | | H4H1276S | |
| injection | Mean | SEM | Mean | SEM |
| −7 | 450.67 | 25.68 | 479.33 | 13.76 |
| 1 | 497.50 | 37.77 | 386.33 | 28.59 |
| 4 | 395.00 | 14.37 | 281.20 | 20.83 |
| 7 | 447.33 | 22.18 | 295.50 | 12.86 |
| 11 | 463.80 | 36.01 | 398.03 | 23.13 |

TABLE 23

| | Non-HDL cholesterol (mg/dL) | | | |
|---|---|---|---|---|
| Days after | Control (hIgG4) | | H4H1276S | |
| injection | Mean | SEM | Mean | SEM |
| −7 | 435.87 | 25.59 | 464.53 | 13.97 |
| 1 | 476.30 | 37.29 | 371.25 | 28.65 |
| 4 | 375.61 | 14.51 | 266.26 | 21.19 |
| 7 | 427.66 | 21.45 | 280.75 | 12.55 |
| 11 | 442.27 | 34.19 | 379.55 | 22.31 |

TABLE 24

| | LDL cholesterol (mg/dL) | | | |
|---|---|---|---|---|
| Days after | Control (hIgG4) | | H4H1276S | |
| injection | Mean | SEM | Mean | SEM |
| −7 | 14.27 | 1.63 | 14.87 | 0.90 |
| 1 | 17.42 | 2.94 | 11.23 | 1.81 |
| 4 | 10.28 | 1.52 | 6.62 | 0.83 |
| 7 | 11.82 | 1.40 | 6.32 | 0.45 |
| 11 | 13.90 | 2.54 | 10.21 | 1.14 |

TABLE 25

| | HDL cholesterol (mg/dL) | | | |
|---|---|---|---|---|
| Days after | Control (hIgG4) | | H4H1276S | |
| injection | Mean | SEM | Mean | SEM |
| −7 | 14.80 | 0.37 | 14.80 | 0.54 |
| 1 | 21.20 | 1.00 | 15.08 | 0.53 |
| 4 | 19.33 | 0.94 | 14.53 | 0.75 |
| 7 | 19.77 | 0.78 | 14.58 | 0.72 |
| 11 | 21.53 | 1.89 | 18.48 | 1.00 |

Single administration of H4H1276S to apoE^{-/-} mice at 10 mg/kg led to ˜72% (mean) reduction in circulating triglycerides (Table 21) and ˜46% (mean) reduction in LDL cholesterol (Table 24) 7 days after the antibody administration (compared to the isotype-matched control Ab, i.e., hlgG4). The administration of H4H1276S also led to a reduction in total cholesterol (Table 22) and non-HDL cholesterol (Table 23).

Levels of circulating H4H1276S (Serum Ab) were also determined using a standard ELISA assay. Briefly, plates were coated with a goat anti-human Fc antibody (Sigma-Aldrich) to capture Serum Ab. Serum was then added to the plates and captured antibodies were detected by chemiluminescence using a horseradish peroxidase (HRP) conjugated goat anti-human IgG antibody (Sigma-Aldrich). Results, expressed as (mean±SEM), are shown in Table 26 (Control: mice that received an isotype-matched control Ab, i.e., hlgG4).

TABLE 26

| | Serum Ab (μg/mL) | | | |
|---|---|---|---|---|
| | Control (hIgG4) | | H4H1276S | |
| Days after | (10 mg/kg) | | (10 mg/kg) | |
| injection | Mean | SEM | Mean | SEM |
| 1 | 89.98 | 16.70 | 115.29 | 19.75 |
| 4 | 67.18 | 2.38 | 86.61 | 5.32 |
| 7 | 58.52 | 2.00 | 39.85 | 6.91 |
| 12 | 43.26 | 1.76 | 3.18 | 2.64 |

### Example 9. In Vivo Effect of H4H1276S on Circulating Lipid Levels in Hyperlipidemic Ldlr^{-/-} Mice

The effect of the anti-hANGPTL3 antibody H4H1726S on serum lipid levels was determined in Ldlr^{-/-} mice. These mice are hyperlipemic with a majority of circulating cholesterol found in the form of LDL due to the lack of LDLR, the major receptor for LDL cholesterol uptake.

Mice were pre-bled 7 days before the experiment and put into groups of six mice. The antibodies, H4H1726S and isotype-matched (hlgG4) negative control, were administered at 10 mg/kg dose by subcutaneous injection on day 0 of the study. Mice were bled after 4 hours of fasting at days 1, 4, 7 and 11 after antibody injection and serum lipids levels (triglycerides, total cholesterol, non-HDL cholesterol, LDL cholesterol and HDL cholesterol) were determined using an ADVIA® 1800 Chemistry System (Siemens) clinical chemistry analyzer. Averages were calculated for each time point for each antibody. Results, expressed as (mean±SEM) of serum lipids concentration (triglycerides, total cholesterol, non-HDL cholesterol, LDL cholesterol and HDL cholesterol), are shown in Tables 27-31, respectively. (Control=mice that received an isotype-matched control antibody).

TABLE 27
Serum triglycerides (mg/dL)

| | Antibody | | | |
|---|---|---|---|---|
| | Control | | H4H1726S | |
| Days after | (10 mg/kg) | | (10 mg/kg) | |
| injection | Mean | SEM | Mean | SEM |
| −7 | 114.50 | 11.08 | 110.83 | 6.89 |
| 1 | 131.50 | 6.18 | 74.17 | 3.30 |
| 4 | 112.67 | 8.94 | 68.00 | 3.91 |
| 7 | 136.67 | 11.55 | 92.67 | 12.16 |
| 11 | 142.33 | 7.10 | 95.83 | 8.67 |

TABLE 28
Total cholesterol (mg/dL)

| | Antibody | | | |
|---|---|---|---|---|
| | Control | | H4H1726S | |
| Days after | (10 mg/kg) | | (10 mg/kg) | |
| injection | Mean | SEM | Mean | SEM |
| −7 | 237.95 | 7.33 | 236.99 | 5.68 |
| 1 | 241.97 | 10.58 | 206.98 | 9.68 |
| 4 | 229.88 | 7.61 | 172.96 | 4.49 |
| 7 | 234.74 | 10.49 | 176.28 | 7.47 |
| 11 | 251.87 | 18.82 | 201.73 | 10.12 |

TABLE 29
Non-HDL cholesterol (mg/dL)

| | Antibody | | | |
|---|---|---|---|---|
| | Control | | H4H1726S | |
| Days after | (10 mg/kg) | | (10 mg/kg) | |
| injection | Mean | SEM | Mean | SEM |
| −7 | 180.81 | 7.47 | 182.79 | 5.93 |
| 1 | 184.35 | 10.22 | 155.93 | 8.74 |
| 4 | 175.13 | 7.26 | 130.79 | 4.66 |
| 7 | 174.84 | 9.26 | 126.56 | 6.63 |
| 11 | 190.00 | 17.07 | 145.43 | 7.34 |

TABLE 30
LDL cholesterol (mg/dL)

| | Antibody | | | |
|---|---|---|---|---|
| | Control | | H4H1726S | |
| Days after | (10 mg/kg) | | (10 mg/kg) | |
| injection | Mean | SEM | Mean | SEM |
| −7 | 62.75 | 2.18 | 62.75 | 1.81 |
| 1 | 63.25 | 2.40 | 53.82 | 4.09 |
| 4 | 60.97 | 3.14 | 49.65 | 2.72 |
| 7 | 59.52 | 2.99 | 46.05 | 2.13 |
| 11 | 63.23 | 3.07 | 54.28 | 1.67 |

TABLE 31
HDL cholesterol (mg/dL)

| | Antibody | | | |
|---|---|---|---|---|
| | Control | | H4H1726S | |
| Days after | (10 mg/kg) | | (10 mg/kg) | |
| injection | Mean | SEM | Mean | SEM |
| −7 | 57.13 | 1.56 | 54.20 | 1.89 |
| 1 | 57.62 | 0.88 | 51.05 | 0.98 |
| 4 | 54.75 | 2.23 | 42.17 | 1.89 |
| 7 | 59.90 | 2.51 | 49.72 | 2.35 |
| 11 | 61.87 | 2.48 | 56.30 | 3.43 |

As shown in Tables 27-31, administration of H4H1726S to Ldlr-^{-/-} mice led to a significant reduction in plasma triglycerides with a maximal observed reduction of 44% (based on mean values). Significant reductions in LDL cholesterol (up to 23%), as well as total cholesterol, non-HDL cholesterol and HDL cholesterol, were also observed in H4H1726S-treated subjects. Reduction of LDL cholesterol in mice deficient for the major receptor for LDL cholesterol uptake (LDLR) suggests an LDLR-independent mechanism for LDL cholesterol reduction by ANGPTL3 inhibition.

Levels of circulating H4H1726S (Serum Ab) were also determined using a standard ELISA assay. Briefly, plates were coated with a goat anti-human Fc antibody (Sigma-Aldrich) to capture Serum Ab. Serum was then added to the plates and captured antibodies were detected by chemiluminescence using a horseradish peroxidase (HRP) conjugated goat anti-human IgG antibody (Sigma-Aldrich). Results, expressed as (mean±SEM) of are shown in Table 32. (Control=mice that received an isotype-matched control antibody).

TABLE 32
Serum Ab (μg/mL)

| | Antibody | | | |
|---|---|---|---|---|
| | Control | | H4H1726S | |
| Days after | (10 mg/kg) | | (10 mg/kg) | |
| injection | Mean | SEM | Mean | SEM |
| 1 | 44.59 | 1.95 | 58.79 | 5.95 |
| 4 | 42.28 | 6.12 | 47.21 | 10.24 |
| 7 | 41.76 | 3.87 | 28.88 | 5.97 |
| 11 | 37.25 | 6.85 | 21.02 | 4.86 |

As shown in Table 32, serum levels of H4H1726S decreased to about 21 μg/mL by day 11 following injection of mice with 10 mg/kg of antibody.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

We claim:

1. An isolated human antibody or antigen-binding fragment thereof that specifically binds human angiopoietin-like protein 3 (hANGPTL3) of SEQ ID NO: 161 and neutralizes, reduces, or interferes with, at least one activity of hANGPTL3.

2. The antibody or fragment of claim 1, wherein the antibody or fragment thereof binds to an epitope situated within the N-terminal coiled-coil region at residues 17 to 209 of SEQ ID NO: 161.

3. The antibody or fragment of claim 2, wherein the antibody or fragment thereof does not bind to the ANGPTL3 peptide of SEQ ID NO: 170.

4. The antibody or fragment of any one of claim 2, wherein the epitope is situated within residues 17 to 200, 17 to 100, 17 to 70, 17 to 65, 17 to 60, 17 to 57, 17 to 50, 40 to 200, 40 to 100, 40 to 70, 50 to 200, 50 to 100, 50 to 70, 58 to 200, 58 to 100, 58 to 70, 58 to 68, or 61 to 66, of SEQ ID NO: 161.

5. The antibody or fragment of any one of claim 1, wherein the antibody or fragment is a chimeric or fully human antibody or antibody fragment.

6. The antibody or fragment of any one of claim 1, comprising a heavy chain variable region (HCVR) selected from the group consisting of SEQ ID NO:2, 18, 34, 50, 66, 82, 98, 114, 130, 146 and 180; and/or a light chain variable region (LCVR) selected from the group consisting of SEQ ID NO: 10, 26, 42, 58, 74, 90, 106, 122, 138, 154 and 188.

7. The antibody or fragment of claim 6, wherein the antibody or fragment thereof comprises a HCVR and LCVR sequence pair (HCVR/LCVR) selected from the group consisting of SEQ ID NO: 2/10, 18/26, 34/42, 50/58, 66/74, 82/90, 98/106, 114/122, 130/138, 146/154 and 180/188.

8. The antibody or fragment of any one of claims 1, wherein the antibody or fragment comprises heavy chain complementarity determining region (CDR) sequences, HCDR1, HCDR2 and HCDR3, and light chain CDR sequences, LCDR1, LCDR2 and LCDR3, contained within a heavy chain variable region (HCVR) and light chain variable region (LCVR) sequence pair (HCVR/LCVR) selected from the group consisting of SEQ ID NO:2/10, 18/26, 34/42, 50/58, 66/74, 82/90, 98/106, 114/122, 130/138, 146/154 and 180/188.

9. The antibody or fragment of claim 8, wherein the HCDR1/HCDR2/HCDR3 sequence combination is selected from the group consisting of SEQ ID NO:4/6/8, 20/22/24, 36/38/40, 52/54/56, 68/70/72, 84/86/88, 100/102/104, 116/118/120, 132/134/136, 148/150/152 and 182/184/186; and/or the LCDR1/LCDR2/LCDR3 sequence combination selected from the group consisting of SEQ ID NO: 12/AAS/16, 28/KAS/32, 44/AAS/48, 60/KAS/64, 76/KAS/80, 92/TTS/96, 108/PAS/112, 124/TAS/128, 140/KVS/144, 156/VAA/160 and 190/KAS/194.

10. The antibody or fragment of claim 9, wherein the antibody or fragment comprises a HCDR1/HCDR2/HCDR3/LCDR1/LCDR2/LCDR3 sequence combination selected from the group consisting of 4/6/8/12/AAS/16, 20/22/24/28/KAS/32, 36/38/40/44/AAS/48, 52/54/56/60/KAS/64, 68/70/72/76/KAS/80, 84/86/88/92/TTS/96, 100/102/104/108/PAS/112, 116/118/120/124/TAS/128, 132/134/136/140/KVS/144, 148/150/152/156/VAA/160 and 182/184/186/190/KAS/194.

11. An antibody or antigen-binding fragment thereof which competes for binding to hANGPTL3 with, or which binds the same epitope on hANGPTL3 as, an antibody or antigen-binding fragment comprising a heavy chain and light chain complementarity determining region (CDR) sequence combination selected from the group consisting of 4/6/8/12/AAS/16, 20/22/24/28/KAS/32, 36/38/40/44/AAS/48, 52/54/56/60/KAS/64, 68/70/72/76/KAS/80, 84/86/88/92/TTS/96, 100/102/104/108/PAS/112, 116/118/120/124/TAS/128, 132/134/136/140/KVS/144, 148/150/152/156/VAA/160 and 182/184/186/190/KAS/194.

12. The antibody or antigen-binding fragment of claim 1, wherein the antibody or antigen-binding fragment cross-reacts with cynomolgus monkey ANGPTL3.

13. The antibody or antigen-binding fragment of claim 1, wherein the antibody or antigen-binding fragment cross-reacts with mouse or rat ANGPTL3.

14. The antibody or antigen-binding fragment of claim 1, wherein the antibody or fragment cross-reacts with any of cynomolgus monkey ANGPTL3, mouse ANGPTL3, and rat ANGPTL3.

15. The antibody or antigen-binding fragment of claim 1, wherein the antibody fragment is a single chain antibody, an Fab, or an F(ab′)2.

16. An isolated nucleic acid molecule encoding the antibody or antigen-binding fragment of claim 1.

17. An expression vector comprising the nucleic acid molecule of claim 16.

18. An isolated host cell comprising the expression vector of claim 17.

19. A method of producing an anti-hANGPTL3 antibody or antigen-binding fragment thereof, comprising growing the host cell of claim 18 under conditions permitting production of the antibody or fragment thereof, and recovering the antibody or fragment thereof so produced.

20. A pharmaceutical composition comprising the antibody or antigen-binding fragment of claim 1, and a pharmaceutically acceptable carrier.

21. The pharmaceutical composition of claim 20, wherein the antibody or fragment thereof comprises a heavy chain and light chain complementarity determining region (CDR) sequence combination of SEQ ID NO:68/70/72/76/KAS/80, or a heavy chain variable region and a light chain variable region HCVR/LCVR pair comprising SEQ ID NO:66/74.

22. A method for preventing or treating a disease or disorder which is prevented, ameliorated, improved or inhibited by reduction or inhibition of ANGPTL3 activity, the method comprising administering to a subject in need thereof a therapeutically effective amount of the pharmaceutical composition of claim 20.

23. The method of claim 22, further comprising administering to the subject one or more additional therapeutic agents selected from the group consisting of an inhibitor of HMG-CoA reductase; an inhibitor of cholesterol uptake or bile acid re-absorption, or both; an agent which increases lipoprotein catabolisman agent which reduces the number of non-fatal heart attacks; and an activator of the LXR transcription factor.

24. The method of claim 22, further comprising administering to the subject one or more additional therapeutic agents selected from the group consisting of a statin, niacin, fibrate, anti-hANGPTL4 antibody and anti-PCSK9 antibody.

25. The method of claim 22, wherein the disease or disorder is selected from the group consisting of hypertriglyceridemia, hypercholesterolemia, chylomicronemia, atherogenic dyslipidemia, a cardiovascular disease or disorder, acute pancreatitis, nonalcoholic steatohepatitis (NASH), diabetes and obesity.

26. A method of enhancing the clearance of hANGPTL3 from the circulation of a subject in need thereof, the method comprising administering to the subject at least two antibodies or antigen-binding fragments thereof that do not compete with one another for binding to hANGPTL3 and do not block at least one activity of hANGPTL3.

27. The method of claim 26, wherein the at least two antibodies or antigen-binding fragments do not block lipoprotein lipase (LPL) activity of ANGPTL3.

28. The method of claim 27, wherein the two antibodies or antigen-binding fragments thereof comprise HCVR/LCVR amino acid sequence pairs selected from the group consisting of:
(i) SEQ ID NO:82/90 and 180/188;
(ii) SEQ ID NO:82/90 and 18/26;
(iii) SEQ ID NO:114/122 and 180/188; and
(iv) SEQ ID NO:114/122 and 18/26.
